(19) United States
(12) Patent Application Publication
(10) Pub. No.: US 2008/0076785 A1
(43) Pub. Date: Mar. 27, 2008

(54) 7-Aminomethyl-1,2,4-Triazolo[1,5-A]Pyrimidine Compounds And Their Use For Controlling Pathogenic Fungi

(76) Inventors: Carsten Blettner, Hong Kong (CN); Jordi Tormo i Blasco, Laudenbach (DE); Bernd Muller, Frankenthal (DE); Markus Gewehr, Kastellaun (DE); Wassilios Grammenos, Ludwigshafen (DE); Thomas Grote, Wachenheim (DE); Udo Hunger, Mainz (DE); Joachim Rheinheimer, Ludwigshafen (DE); Peter Schafer, Ottersheim (DE); Frank Schieweck, Hessheim (DE); Anja Schwogler, Mannheim (DE); Oliver Wagner, Neustadt (DE); John-Bryan Speakman, Bobenheim (DE); Thorsten Jabs, Hassloch (DE); Siegfried Strathmann, Limburgerhof (DE); Ulrich Schofl, Bruhl (DE); Maria Scherer, Landau (DE); Reinhard Stierl, Freinsheim (DE)

Correspondence Address: BIRCH STEWART KOLASCH & BIRCH, PO BOX 747, FALLS CHURCH, VA, 22040-0747, US

(21) Appl. No.: 11/663,982

(22) PCT Filed: Sep. 27, 2005
(86) PCT No.: PCT/EP05/10440

§ 371 Date: Mar. 28, 2007

(30) Foreign Application Priority Data
Sep. 28, 2004 (DE) 10 2004 047 051.0

(51) Int. Cl. A01N 43/54; A01P 3/00; C07D 239/70

(52) U.S. Cl. 514/259.31; 544/263

(57) ABSTRACT

The invention relates to 7-aminomethyl-1,2,4-triazolo[1,5-a]pyrimidine compounds of general formula (I) and the agriculturally compatible salts of said compounds. In said formula: R1 and R2 represent hydrogen, alkyl, haloalkyl, alkoxy, cycloalkyl, bicycloalkyl, halocycloalkyl, alkenyl, alkadienyl, haloalkenyl, cycloalkenyl, halocycloalkenyl, alkynyl, haloalkynyl or phenyl, naphthyl, or a 5- or 6-membered saturated, partially unsaturated or aromatic heterocycle, containing 1 to 4 heteroatoms from the group containing O, N or S, or R1 and R2, together with the nitrogen atom to which they are bonded, can also form a five- or six-membered heterocyclyl or heteroaryl, which is bonded by means of N and can contain 1 to 3 additional heteroatoms from the group containing O, N and S as the ring member and can be substituted according to the description; R3 and R4 represent hydrogen, alkyl, haloalkyl, haloalkoxy, alkoxy, or alkoxyalkyl; X represents halogen, cyano, a 5- or 6-membered saturated, partially unsaturated or aromatic heterocycle, containing 1, 2 or 3 heteroatoms from the group containing O, N or S as the ring member, alkyl, alkoxy, alkenyl or alkynyl, whereby the latter 4 groups can be substituted according to the description; L is defined as cited in the claims and the description; and m represents 0, 1, 2, 3, 4 or 5. The invention also relates to the use of the triazolopyrimidine compounds of general formula (I) and their agriculturally compatible salts for controlling plant-pathogenic fungi, to a method for controlling plant-pathogenic fungi, containing at least one compound of general formula (I) and/or an agriculturally compatible salt of said compound and at least one liquid or solid carrier.

The present invention relates to novel 7-aminomethyl-1,2,4-triazolo[1,5-a]pyrimidine compounds and to their use for controlling harmful fungi, and to crop protection compositions comprising at least one such compound as active ingredient.

EP-A 71 792, EP-A 550 113, EP-A 834 513 and WO-A 98/46608 describe fungicidally active 1,2,4-triazolo[1,5-a]pyrimidines carrying an optionally substituted phenyl ring in the 6-position, a halogen atom in the 5-position and an amino group in the 7-position.

WO-A-99/41255 describes fungicidally active 1,2,4-triazolo[1,5-a]pyrimidine compounds carrying a halogen atom, a cyano, haloalkoxy or alkoxy group in the 5-position and an optionally substituted aliphatic, cycloaliphatic or aromatic radical in the 7-position.

WO 03/004465 describes fungicidally active 1,2,4-triazolo[1,5-a]pyrimidine compounds carrying an optionally substituted aliphatic, cycloaliphatic or aromatic radical in the 7-position and an optionally substituted alkyl-, alkenyl- or alkynyl group in the 5-position.

Some of the 1,2,4-triazolo[1,5-a]pyrimidines known from the prior art are, with respect to their fungicidal action, not satisfactory, or they have unwanted properties, such as poor crop plant compatibility.

Accordingly, it is an object of the present invention to provide novel compounds having improved fungicidal activity and/or better crop plant compatibility.

Surprisingly, this object is achieved by 1,2,4-triazolo[1,5-a]pyrimidines of the formula I 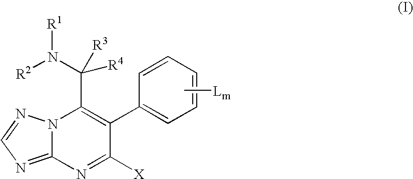
where the index m and the substituents R¹, R², R³, R⁴, X and L are as defined below:
R¹, R² independently of one another are hydrogen, C₁-C₈-alkyl, C₁-C₈-haloalkyl, C₁-C₈-alkoxy, C₃-C₈-cycloalkyl, C₅-C₁₀-bicycloalkyl, C₃-C₈-halocycloalkyl, C₂-C₈-alkenyl, C₄-C₁₀-alkadienyl, C₂-C₈-haloalkenyl, C₃-C₆-cycloalkenyl, C₃-C₆-halocycloalkenyl, C₂-C₈-alkynyl, C₂-C₈-haloalkynyl or phenyl, naphthyl, or a five- or six-membered saturated, partially unsaturated or aromatic heterocycle which contains one, two, three or four heteroatoms from the group consisting of O, N and S,
R¹ and R² together with the nitrogen atom to which they are attached may also form a five- or six-membered heterocyclyl or heteroaryl which is attached via N and may contain one, two or three further heteroatoms from the group consisting of O, N and S as ring members and/or may carry one or more substituents from the group consisting of halogen, C₁-C₆-alkyl, C₁-C₆-haloalkyl, C₂-C₆-alkenyl, C₂-C₆-haloalkenyl, C₁-C₆-alkoxy, C₁-C₆-alkoxycarbonyl, C₁-C₆-haloalkoxy, C₃-C₆-alkenyloxy, C₃-C₆-haloalkenyloxy and/or in which two substituents attached to adjacent ring atoms may be C₁-C₆-alkylene, oxy-C₂-C₄-alkylene or oxy-C₁-C₃-alkyleneoxy;

R¹ and/or R² may carry one, two, three or four identical or different groups R^{a}:
R^{a} is halogen, cyano, nitro, hydroxyl, carboxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₁-C₆-alkylaminocarbonyl, di-C₁-C₆-alkylaminocarbonyl, C₂-C₈-alkenyl, C₄-C₁₀-alkadienyl, C₂-C₈-haloalkenyl, C₃-C₈-cycloalkenyl, C₂-C₆-alkenyloxy, C₃-C₆-haloalkenyloxy, C₂-C₆-alkynyl, C₂-C₆-haloalkynyl, C₃-C₆-alkynyloxy, C₃-C₆-haloalkynyloxy, C₃-C₆-cycloalkoxy, C₃-C₆-cycloalkenyloxy, oxy-C₁-C₃-alkyleneoxy, phenyl, naphthyl, a five-, six-, seven-, eight-, nine- or ten-membered saturated, partially unsaturated or aromatic heterocycle which contains one, two, three or four heteroatoms from the group consisting of O, N and S,
   where the aliphatic, alicylic or aromatic groups in R^{a} for their part may be partially or fully halogenated or may carry one, two or three groups R^{b}:
   R^{b} is halogen, cyano, nitro, hydroxyl, mercapto, amino, carboxyl, aminocarbonyl, aminothiocarbonyl, alkyl, haloalkyl, alkenyl, alkadienyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothiocarbonyl, where the alkyl groups in these radicals contain 1 to 6 carbon atoms and the alkenyl, alkadienyl or alkynyl groups mentioned in these radicals contain 2 to 8 carbon atoms;
      and/or one, two or three of the following radicals:
      cycloalkyl, bicycloalkyl, cycloalkoxy, heterocyclyl, heterocyclyloxy, where the cyclic systems contain 3 to 10 ring members; aryl, aryloxy, arylthio, aryl-C₁-C₆-alkoxy, aryl-C₁-C₆-alkyl, hetaryl, hetaryloxy, hetarylthio, where the aryl radicals preferably contain 6, 7, 8, 9 or 10 ring members and the hetaryl radicals 5 or 6 ring members, where the cyclic systems may be partially or fully halogenated or substituted by alkyl or haloalkyl groups;
R³, R⁴ independently of one another are hydrogen, C₁-C₈-alkyl, C₁-C₈-haloalkyl, C₁-C₈-haloalkoxy, C₁-C₈-alkoxy, C₁-C₈-haloalkoxy or C₁-C₈-alkoxy-C₁-C₈-alkyl, where the 6 lastmentioned radicals may carry one, two, three or four identical or different radicals R^{c}, where R^{c} has the meanings given for R^{a};
X is halogen, cyano, a 5- or 6-membered saturated, partially unsaturated or aromatic heterocycle which contains 1, 2 or 3 heteroatoms from the group consisting of O, N and S as ring members, C₁-C₄-alkyl, C₁-C₄-alkoxy, C₂-C₈-alkenyl or C₂-C₈-alkynyl, where the four lastmentioned radicals may be partially or fully halogenated and/or may carry one, two or three substituents selected from the group consisting of nitro, cyano, C₁-C₂-alkoxy, C₁-C₄-alkoxycarbonyl, amino, C₁-C₄-alkylamino and di-C₁-C₄-alkylamino and where the 5- or 6-membered heterocycle may carry one or more substituents from the group consisting of halogen, C₁-C₆-alkyl, C₁-C₆-haloalkyl, C₂-C₆-alkenyl, C₂-C₆-haloalkenyl, C₁-C₆-alkoxy, C₁-C₆-alkoxycarbonyl, C₁-C₆-haloalkoxy, C₃-C₆-alkenyloxy, C₃-C₆-haloalkenyloxy, (exo)-C₁-C₆-alkylene and oxy-C₁-C₃-alkyleneoxy;
L is halogen, cyano, hydroxyl, cyanato (OCN), nitro, C₁-C₈-alkyl, C₂-C₁₀-alkenyl, C₂-C₁₀-alkynyl, C₁-C₆-haloalkyl, C₂-C₁₀-haloalkenyl, C₁-C₆-alkoxy, C₂-C₁₀-alkenyloxy, C₂-C₁₀-alkynyloxy, C₁-C₆-haloalkoxy, C₃-C₆-cycloalkyl, C₃-C₆-cycloalkenyl, C₃-C₆-cycloalkoxy, C₁-C₈-alkoxycarbonyl, C₂-C₁₀-alkenyloxycarbonyl, C₂-C₁₀-alkynyloxycarbonyl, aminocarbonyl, C₁-C₈-alkylaminocarbonyl, di-(C₁-C₈-)-alkylaminocarbonyl, C₁-C₈-alkoximinoalkyl, C₂-C₁₀-alkenyloximinocarbonyl, C₂-C₁₀-alkynyloximinoalkyl, C₁-C₈-alkylcarbonyl, C₂-C₁₀-alkenylcarbonyl, C₂-C₁₀-alkynylcarbonyl, C₃-C₆-cycloalkylcarbonyl, a five-, six-, seven-, eight-, nine- or ten-membered saturated, partially unsaturated or aromatic heterocycle which contains one, two, three or four heteroatoms selected from the group consisting of O, N and S, amino, NR⁵R⁶, NR⁵—(C═O)—R⁶, S(═O)ₙA¹, C(═O)A², C(═S)A², a group —C(S)NR⁷R⁸, a group —C(═N—OR⁹)(NR¹⁰R¹¹) or a group —C(═N—NR¹²R¹³)(NR¹⁴R¹⁵),
   where
   R⁵, R⁶ independently of one another are selected from the group consisting of hydrogen, C₁-C₆-alkyl, C₂-C₁₀-alkenyl, C₂-C₁₀-alkynyl, C₃-C₆-cycloalkyl and C₃-C₆-cycloalkenyl, where the 5 lastmentioned radicals may be partially or fully halogenated or may carry one, two, three or four radicals selected from the group consisting of cyano, C₁-C₄-alkoximino, C₂-C₄-alkenyloximino, C₂-C₄-alkynyloximino and C₁-C₄-alkoxy;
   A¹ is hydrogen, hydroxyl, C₁-C₈-alkyl, C₁-C₈-alkylamino or di-(C₁-C₈-alkyl)amino;
   n is 0, 1 or 2;
   A² is C₂-C₈-alkenyl, C₁-C₈-alkoxy, C₁-C₆-haloalkoxy, amino or one of the groups mentioned under A¹;
   R⁷, R⁸, R⁹, R¹⁰, R¹¹, R¹², R¹³, R¹⁴ and R¹⁵ independently of one another are selected from the group consisting of hydrogen, C₁-C₆-alkyl, C₃-C₆-cycloalkyl, C₂-C₆-alkenyl and C₂-C₆-alkynyl, where the four lastmentioned radicals may have one, two, three, four, five or six radicals R^{a}; or
   R⁷ and R⁸, R¹⁰ and R¹¹, R¹² and R¹³ and/or R¹⁴ and R¹⁵ together with the nitrogen atom to which they are attached form a four-, five- or six-membered saturated or partially unsaturated ring which may carry one, two, three or four substituents independently of one another selected from R^{a};
m is 0, 1, 2, 3, 4 or 5;
   and by the agriculturally acceptable salts of the compounds I.

Accordingly, the present invention provides the 7-aminomethyl-1,2-4-triazolo[1,5-a]compounds of the formula I and their agriculturally acceptable salts.

The present invention furthermore provides the use of the 7-aminomethyl-1,2-4-triazolo[1,5-a]compounds of the formula I and their agriculturally acceptable salts for controlling phytopathogenic fungi (=harmful fungi), and a method for controlling phytopathogenic fungi wherein the fungi or the materials, plants, the soil or seeds to be protected against fungal attack are treated with an effective amount of at least one compound of the formula I and/or with an agriculturally acceptable salt of 1.

The present invention furthermore provides a composition for controlling phytopathogenic fungi, which composition comprises at least one compound of the formula I and/or an agriculturally acceptable salt thereof and at least one liquid or solid carrier.

Depending on the substitution pattern, the compounds of the formula I may have one or more centers of chirality, in which case they are present as enantiomer or diastereomer mixtures. The present invention provides both the pure enantiomers or diastereomers and their mixtures. Suitable compounds of the formula I also include all possible stereoisomers (cis/trans isomers) and mixtures thereof.

Agriculturally useful salts are especially the salts of those cations or the acid addition salts of those acids whose cations and anions, respectively, have no adverse effect on the fungicidal action of the compounds I. Suitable cations are thus in particular the ions of the alkali metals, preferably sodium and potassium, of the alkaline earth metals, preferably calcium, magnesium and barium, of the transition metals, preferably manganese, copper, zinc and iron, and also the ammonium ion which, if desired, may carry one to four C₁-C₄-alkyl substituents and/or one phenyl or benzyl substituent, preferably diisopropylammonium, tetramethylammonium, tetrabutylammonium, trimethylbenzylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogen-sulfate, sulfate, dihydrogenphosphate, hydrogenphosphate, phosphate, nitrate, bicarbonate, carbonate, hexafluorosilicate, hexafluorophosphate, benzoate, and the anions of C₁-C₄-alkanoic acids, preferably formate, acetate, propionate and butyrate. They can be formed by reacting I with an acid of the corresponding anion, preferably of hydrochloric acid, hydrobromic acid, sulfuric acid, phosphoric acid or nitric acid.

In the definitions of the variables given in the formulae above, collective terms are used which are generally representative for the substituents in question. The term Cₙ-Cₘ indicates the number of carbon atoms possible in each case in the substituent or substituent moiety in question:

halogen: fluorine, chlorine, bromine and iodine;

alkyl: saturated straight-chain or branched hydrocarbon radicals having 1 to 4, 6 or 8 carbon atoms, for example C₁-C₆-alkyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl and the like;

haloalkyl: straight-chain or branched alkyl groups having 1 to 2, 4, 6 or 8 carbon atoms (as mentioned above), where in these groups some or all of the hydrogen atoms may be replaced by halogen atoms as mentioned above: in particular, C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl;

alkenyl: monounsaturated straight-chain or branched hydrocarbon radicals having 2 to 4, 2 to 6, 2 to 8 or 2 to 10 carbon atoms and a double bond in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl and the like;

alkadienyl: doubly unsaturated straight-chain or branched hydrocarbon radicals having 4 to 10 carbon atoms and two double bonds in any position, for example 1,3-butadienyl, 1-methyl-1,3-butadienyl, 2-methyl-1,3-butadienyl, penta-1,3-dien-1-yl, hexa-1,4-dien-1-yl, hexa-1,4-dien-3-yl, hexa-1,4-dien-6-yl, hexa-1,5-dien-1-yl, hexa-1,5-dien-3-yl, hexa-1,5-dien-4-yl, hepta-1,4-dien-1-yl, hepta-1,4-dien-3-yl, hepta-1,4-dien-6-yl, hepta-1,4-dien-7-yl, hepta-1,5-dien-1-yl, hepta-1,5-dien-3-yl, hepta-1,5-dien-4-yl, hepta-1,5-dien-7-yl, hepta-1,6-dien-1-yl, hepta-1,6-dien-3-yl, hepta-1,6-dien-4-yl, hepta-1,6-dien-5-yl, hepta-1,6-dien-2-yl, octa-1,4-dien-1-yl, octa-1,4-dien-2-yl, octa-1,4-dien-3-yl, octa-1,4-dien-6-yl, octa-1,4-dien-7-yl, octa-1,5-dien-1-yl, octa-1,5-dien-3-yl, octa-1,5-dien-4-yl, octa-1,5-dien-7-yl, octa-1,6-dien-1-yl, octa-1,6-dien-3-yl, octa-1,6-dien-4-yl, octa-1,6-dien-5-yl, octa-1,6-dien-2-yl, deca-1,4-dienyl, deca-1,5-dienyl, deca-1,6-dienyl, deca-1,7-dienyl, deca-1,8-dienyl, deca-2,5-dienyl, deca-2,6-dienyl, deca-2,7-dienyl, deca-2,8-dienyl and the like;

haloalkenyl: unsaturated straight-chain or branched hydrocarbon radicals having 2 to 10 carbon atoms and a double bond in any position (as mentioned above), where in these groups some or all of the hydrogen atoms may be replaced by halogen atoms as mentioned above, in particular by fluorine, chlorine and bromine;

alkynyl: straight-chain or branched hydrocarbon groups having 2 to 4, 2 to 6, 2 to 8 or 2 to 10 carbon atoms and one or two triple bonds in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl and the like;

cycloalkyl: monocyclic saturated hydrocarbon groups having 3 to 8 carbon ring members, such as cyclopropyl, cyclobutyl, cyclopentyl, cyclohexyl, cycloheptyl and cyclooctyl;

cycloalkenyl: monocyclic monounsaturated hydrocarbon groups having 3 to 8, preferably 5 to 6 carbon ring members, such as cyclopenten-1-yl, cyclopenten-3-yl, cyclohexen-1-yl, cyclohexen-3-yl, cyclohexen-4-yl and the like;

bicycloalkyl: a bicyclic hydrocarbon radical having 5 to 10 carbon atoms, such as bicyclo[2.2.1]hept-1-yl, bicyclo[2.2.1]hept-2-yl, bicyclo[2.2.1]hept-7-yl, bicyclo[2.2.2]oct-1-yl, bicyclo[2.2.2]oct-2-yl, bicyclo[3.3.0]octyl, bicyclo[4.4.0]decyl and the like;

C₁-C₄-alkoxy: an alkyl group having 1 to 4 carbon atoms which is attached via oxygen, for example methoxy, ethoxy, n-propoxy, 1-methylethoxy, butoxy, 1-methylpropoxy, 2-methylpropoxy or 1,1-dimethylethoxy;

C₁-C₆-alkoxy: C₁-C₄-alkoxy as mentioned above, and also, for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy;

C₁-C₄-haloalkoxy: a C₁-C₄-alkoxy radical as mentioned above which is partially or fully substituted by fluorine, chlorine, bromine and/or iodine, preferably by fluorine, i.e., for example, OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy;

C₁-C₆-haloalkoxy: C₁-C₄-haloalkoxy as mentioned above, and also, for example, 5-fluoropentoxy, 5-chloropentoxy, 5-bromopentoxy, 5-iodopentoxy, undecafluoropentoxy, 6-fluorohexoxy, 6-chlorohexoxy, 6-bromohexoxy, 6-iodohexoxy or dodecafluorohexoxy;

alkenyloxy: alkenyl as mentioned above which is attached via an oxygen atom, for example C₃-C₆-alkenyloxy, such as, 1-propenyloxy, 2-propenyloxy, 1-methylethenyloxy, 1-butenyloxy, 2-butenyloxy, 3-butenyloxy, 1-methyl-1-propenyloxy, 2-methyl-1-propenyloxy, 1-methyl-2-propenyloxy, 2-methyl-2-propenyloxy, 1-pentenyloxy, 2-pentenyloxy, 3-pentenyloxy, 4-pentenyloxy, 1-methyl-1-butenyloxy, 2-methyl-1-butenyloxy, 3-methyl-1-butenyloxy, 1-methyl-2-butenyloxy, 2-methyl-2-butenyloxy, 3-methyl-2-butenyloxy, 1-methyl-3-butenyloxy, 2-methyl-3-butenyloxy, 3-methyl-3-butenyl, 1,1-dimethyl-2-propenyloxy, 1,2-dimethyl-1-propenyloxy, 1,2-dimethyl-2-propenyloxy, 1-ethyl-1-propenyloxy, 1-ethyl-2-propenyloxy, 1-hexenyloxy, 2-hexenyloxy, 3-hexenyloxy, 4-hexenyloxy, 5-hexenyloxy, 1-methyl-1-pentenyloxy, 2-methyl-1-pentenyloxy, 3-methyl-1-pentenyloxy, 4-methyl-1-pentenyloxy, 1-methyl-2-pentenyloxy, 2-methyl-2-pentenyloxy, 3-methyl-2-pentenyloxy, 4-methyl-2-pentenyloxy, 1-methyl-3-pentenyloxy, 2-methyl-3-pentenyloxy, 3-methyl-3-pentenyloxy, 4-methyl-3-pentenyloxy, 1-methyl-4-pentenyloxy, 2-methyl-4-pentenyloxy, 3-methyl-4-pentenyloxy, 4-methyl-4-pentenyloxy, 1,1-dimethyl-2-butenyloxy, 1,1-dimethyl-3-butenyloxy, 1,2-dimethyl-1-butenyloxy, 1,2-dimethyl-2-butenyloxy, 1,2-dimethyl-3-butenyloxy, 1,3-dimethyl-1-butenyloxy, 1,3-dimethyl-2-butenyloxy, 1,3-dimethyl-3-butenyloxy, 2,2-dimethyl-3-butenyloxy, 2,3-dimethyl-1-butenyloxy, 2,3-dimethyl-2-butenyloxy, 2,3-dimethyl-3-butenyloxy, 3,3-dimethyl-1-butenyloxy, 3,3-dimethyl-2-butenyloxy, 1-ethyl-1-butenyloxy, 1-ethyl-2-butenyloxy, 1-ethyl-3-butenyloxy, 2-ethyl-1-butenyloxy, 2-ethyl-2-butenyloxy, 2-ethyl-3-butenyloxy, 1,1,2-trimethyl-2-propenyloxy, 1-ethyl-1-methyl-2-propenyloxy, 1-ethyl-2-methyl-1-propenyloxyand 1-ethyl-2-methyl-2-propenyloxy;

alkynyloxy: alkynyl as mentioned above which is attached via an oxygen atom, for example C₃-C₆-alkynyloxy, such as 2-propynyloxy, 2-butynyloxy, 3-butynyloxy, 1-methyl-2-propynyloxy, 2-pentynyloxy, 3-pentynyloxy, 4-pentynyloxy, 1-methyl-2-butynyloxy, 1-methyl-3-butynyloxy, 2-methyl-3-butynyloxy, 1-ethyl-2-propynyloxy, 2-hexynyloxy, 3-hexynyloxy, 4-hexynyloxy, 5-hexynyloxy, 1-methyl-2-pentynyloxy, 1-methyl-3-pentynyloxy and the like;

5-, 6-, 7-, 8-, 9- or 10-membered saturated, partially unsaturated or aromatic heterocycle which contains 1, 2, 3 or 4 heteroatoms from the group consisting of oxygen, nitrogen and sulfur:

a five- or six-membered saturated or partially unsaturated heterocycle which contains, as ring members, one, two, three or four heteroatoms selected from the group consisting of oxygen, nitrogen and sulfur: for example monocyclic or partially unsaturated heterocycles (heterocyclyl) containing, in addition to carbon ring members, one to three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-lsothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl and the corresponding -ylidene radicals;
a seven-membered saturated or partially unsaturated heterocycle which contains, as ring members, one, two, three or four heteroatoms selected from the group consisting of oxygen, nitrogen and sulfur: for example mono- and bicyclic heterocycles having 7 ring members which contain, in addition to carbon ring members, one to three nitrogen atoms and/or one oxygen or sulfur atom or one or two oxygen and/or sulfur atoms, for example tetra- and hexahydroazepinyl, such as 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or -7-yl, 2,3,4,7-tetrahydro[1H]azepin-1-, -2-, -3-, -4-, -5-, -6- or -7-yl, 2,3,6,7-tetrahydro[1H]azepin-1-, -2-, -3-, -4-, -5-, -6- or -7-yl, hexahydroazepin-1-, -2-, -3- or -4-yl, tetra- and hexahydrooxepinyl, such as 2,3,4,5-tetrahydro[1H]oxepin-2-, -3-, -4-, -5-, -6- or -7-yl, 2,3,4,7-tetrahydro[1H]oxepin-2-, -3-, -4-, -5-, -6- or -7-yl, 2,3,6,7-tetrahydro[1H]oxepin-2-, -3-, -4-, -5-, -6- or -7-yl, hexahydroazepin-1-, -2-, -3- or -4-yl, tetra- and hexahydro-1,3-diazepinyl, tetra- and hexahydro-1,4-diazepinyl, tetra- and hexahydro-1,3-oxazepinyl, tetra- and hexahydro-1,4-oxazepinyl, tetra- and hexahydro-1,3-dioxepinyl, tetra- and hexahydro-1,4-dioxepinyl and the corresponding -ylidene radicals;
a five- or six-membered aromatic heterocycle which contains one, two, three or four heteroatoms from the group consisting of oxygen, nitrogen and sulfur: mono- or bicyclic heteroaryl, for example 5-membered heteroaryl which is attached via carbon and contains one to three nitrogen atoms or one or two nitrogen atoms and a sulfur or oxygen atom as ring members, such as 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl; 5-membered heteroaryl which is attached via nitrogen and contains one to three nitrogen atoms as ring memberes, such as pyrrol-1-yl, pyrazol-1-yl, imidazol-1-yl, 1,2,3-triazol-1-yl and 1,2,4-triazol-1-yl; 6-membered heteroaryl which contains one to three nitrogen atoms as ring members, such as pyridin-2-yl, pyridin-3-yl, pyridin-4-yl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl;
   alkylene: divalent unbranched chains of 1 to 6 CH₂ groups, for example CH₂, CH₂CH₂, CH₂CH₂CH₂, CH₂CH₂CH₂CH₂, CH₂CH₂CH₂CH₂CH₂ and CH₂CH₂CH₂CH₂CH₂CH₂;
   oxyalkylene: divalent unbranched chains of 2 to 4 CH₂ groups, where one valency is attached to the skeleton via an oxygen atom, for example OCH₂CH₂, OCH₂CH₂CH₂ and OCH₂CH₂CH₂CH₂;
   oxyalkylenoxy: divalent unbranched chains of 1 to 3 CH₂ groups, where both valencies are attached to the skeleton via an oxygen atom, for example OCH₂O, OCH₂CH₂O and OCH₂CH₂CH₂O.

With a view to the activity of the compounds of the formula I according to the invention as fungicides, the index m and the substituents R¹, R², R³, R⁴, X and L independently of one another and preferably in combination particularly preferably have the meanings given below:

Preference is given to compounds of the formula I in which at least one of the radicals R¹ or R² is different from hydrogen.

Particular preference is given to compounds of the formula I in which R¹ is C₁-C₆-alkyl, C₂-C₆-alkenyl, C₂-C₆-alkynyl or C₁-C₈-haloalkyl.

Particular preference is likewise given to compounds of the formula I in which R¹ is a group B: 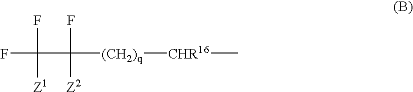
in which
Z¹ is hydrogen, fluorine or C₁-C₆-fluoroalkyl,
Z² is hydrogen or fluorine, or Z¹ and Z² together form a double bond;
q is 0 or 1; and
R¹⁶ is hydrogen or methyl.

Moreover, particular preference is given to compounds of the formula I in which R¹ is C₃-C₆-cycloalkyl which may be substituted by C₁-C₄-alkyl.

Preference is given to compounds of the formula I in which R¹ is different from hydrogen and R² is hydrogen. In this case, R¹ has preferably one of the meanings mentioned as being preferred or particularly preferred.

Preference is likewise given to compounds of the formula I in which R¹ and R² are different from hydrogen. Preferred are compounds of the formula I in which R² is methyl or ethyl. In this case, R¹ has preferably one of the meanings mentioned as being preferred or particularly preferred.

Particular preference is furthermore given to compounds of the formula I in which R¹ and R² together with the nitrogen atom to which they are attached form a saturated heterocyclyl as defined above and in particular a piperidinyl, morpholinyl or thiomorpholinyl ring, especially a piperidinyl ring. In this case, heterocyclyl is unsubstituted or substituted in the manner described above, especially by 1, 2 or 3 substituents R^{a}, preferred substituents on heterocyclyl being selected from the group consisting of halogen, C₁-C₄-alkyl and C₁-C₄-haloalkyl. Very particularly preferred are compounds I in which R¹ and R² together with the nitrogen atom to which they are attached form a 4-methylpiperidine ring or a 3,4-dimethylpiperidine ring.

The invention furthermore particularly preferably provides compounds I in which R¹ and R² together with the nitrogen atom to which they are attached form a 5- or 6-membered heteroaryl as defined above which may be unsubstituted or substituted, preferably by 1, 2 or 3 groups R^{a}. In this case, the group NR¹R² forms in particular a pyrazol ring, which, if appropriate, is substituted in the manner described above and especially by 1 or 2 of the following radicals: halogen, C₁-C₄-alkyl or C₁-C₄-haloalkyl, in particular by 2 methyl groups or 2 trifluoromethyl groups in the 3,5-position.

Particularly preferred are compounds of the formula I, in which R¹ is selected from the group consisting of: CH(CH₃)—CH₂CH₃, CH(CH₃)—CH(CH₃)₂, CH(CH₃)—C(CH₃)₃, CH(CH₃)—CF₃, CH₂C(CH₃)═CH₂, CH₂CH═CH₂, cyclopentyl or cyclohexyl; and R² is hydrogen or methyl; as well as compounds I, in which R¹ and R² together are —(CH₂)₂CH(CH₃)(CH₂)₂—, —(CH₂)₂CH(CF₃)(CH₂)₂— or —(CH₂)₂—O—(CH₂)₂—.

Preference is given to compounds of the formula I, in which R³ and R⁴ independently of one another are hydrogen, C₁-C₄-alkyl or C₁-C₄-alkoxy, particularly preferably hydrogen, C₁-C₂-alkyl such as methyl or ethyl or C₁-C₂-alkoxy such as methoxy or ethoxy.

Particular preference is given to compounds I, in which at least one of the radicals R³ or R⁴ is hydrogen and the other radical R³ or R⁴ has the meanings mentioned above and is in particular hydrogen, methyl or ethyl.

Preferred among the compounds of the formula I are furthermore those in which X is halogen, cyano, C₁-C₄-alkyl, C₁-C₄-haloalkyl, C₁-C₄-alkoxy or C₁-C₂-haloalkoxy. Especially preferred are compounds of the formula I in which X is halogen, C₁-C₂-alkyl, cyano or C₁-C₂-alkoxy, such as chlorine, methyl, cyano, methoxy or ethoxy. X is in particular halogen and especially chlorine.

In the formula l, m is preferably 1, 2, 3 or 4 and in particular 1, 2 or 3.

Preferred meanings of L are: halogen, cyano, nitro, C₁-C₆-alkyl, C₁-C₆-haloalkyl, C₁-C₆-alkoxy, a group C(S)A² or a group C(O)A², where A² has the meanings mentioned above and is preferably C₁-C₄-alkoxy, NH₂, C₁-C₄-alkylamino or di-C₁-C₄-alkylamino. Especially preferred are compounds of the formula I in which the radicals L independently of one another are selected from the group consisting of fluorine, chlorine, bromine, cyano, nitro, C₁-C₄-alkyl, C₁-C₄-haloalkyl, C₁-C₄-alkoxy and C₁-C₄-alkoxycarbonyl, particularly preferably fluorine, chlorine, C₁-C₂-alkyl, such as methyl or ethyl, C₁-C₂-fluoroalkyl, such as trifluoromethyl, C₁-C₂-alkoxy, such as methoxy or C₁-C₂-alkoxycarbonyl, such as methoxycarbonyl. Moreover, preference is given to compounds of the formula I in which at least one radical L is located in the ortho-position to the point of attachment to the triazolopyrimidine skeleton. A radical L located in the ortho-position is preferably selected from the group of the halogens, especially fluorine or chlorine.

In particular, m is 2 or 3 and at least one radical L is located in the ortho-position to the point of attachment to the triazolopyrimidine skeleton. Among these, preference is given to compounds of the formula I in which one radical L is located in the 2-position and a further radical L is located in the 5-position to the point of attachment to the triazolopyrimidine skeleton. Among these, preference is likewise given to compounds of the formula I in which one radical L is located in the 2-position and a further radical L is located in the 4-position to the point of attachment to the triazolopyrimidine skeleton. Among these, preference is likewise given to compounds of the formula I in which one radical L is located in the 2-position and a further radical L is located in the 6-position to the point of attachment to the triazolopyrimidine skeleton.

Particular preference is given in particular to those compounds of the formula I in which the phenyl group substituted by Lₘ is the group C 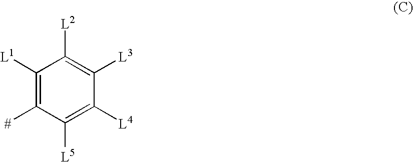
in which # is the point of attachment to the triazolopyrimidine skeleton and
L¹ is fluorine, chlorine, trifluoromethyl or methyl;
L² is hydrogen, chlorine or fluorine;
L³ is hydrogen, CN, nitro, fluorine, chlorine, C₁-C₄-alkyl, C₁-C₄-alkoxy, a group C(S)A^{2a} or a group C(O)A^{2a} in which A^{2a} is C₁-C₄-alkoxy, NH₂, C₁-C₄-alkylamino or di-C₁-C₄-alkylamino;
L⁴ is hydrogen, chlorine or fluorine; and
L⁵ is hydrogen, fluorine, chlorine or C₁-C₄-alkyl.

Besides this, R¹ and R⁶ independently of one another are preferably hydrogen or C₁-C₄-alkyl.

In the radical —C(S)N⁷R⁸, R⁷ and R⁸ independently of one another are preferably selected from the group consisting of hydrogen and C₁-C₆-alkyl, in particular hydrogen and C₁-C₄-alkyl, such as methyl, ethyl, n-propyl or isopropyl, especially hydrogen.

R⁹ is preferably hydrogen or C₁-C₆-alkyl.

R¹⁰ and R¹¹ independently of one another are preferably hydrogen or C₁-C₆-alkyl.

R¹², R¹³, R¹⁴ and R's independently of one another are preferably selected from the group consisting of hydrogen and C₁-C₆-alkyl.

Furthermore, A¹ is preferably hydrogen, C₁-C₆-alkyl or amino. The index n is preferably 0, 1 or 2.

A² is preferably C₁-C₄-alkoxy, NH₂, C₁-C₄-alkylamino or di-C₁-C₄-alkylamino.

A preferred embodiment of the invention relates to compounds of the formula I.1 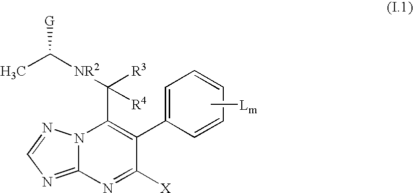
in which
G is C₂-C₆-alkyl, in particular ethyl, n- and i-propyl, n-butyl, sec-butyl, tert-butyl, and C₁-C₄-alkoxymethyl, in particular ethoxymethyl, or C₃-C₆-cycloalkyl, in particular cyclopentyl or cyclohexyl;
R² is hydrogen or methyl;
R³ and R⁴ independently of one another are hydrogen or C₁-C₄-alkyl;
X is chlorine, methyl, cyano, methoxy or ethoxy;
L is halogen, cyano, nitro, C₁-C₆-alkyl, C₁-C₆-haloalkyl, C₁-C₆-alkoxy, a group C(S)A² or a group C(O)A² in which A² is C₁-C₄-alkoxy, NH₂, C₁-C₄-alkylamino or di-C₁-C₄-alkylamino; and
m is 0, 1, 2, 3, 4 or 5.

A further preferred embodiment of the invention relates to compounds in which R¹ and R² together with the nitrogen atom, to which they are attached, form a five- or six-membered heterocyclyl or heteroaryl which is attached via N and which may contain a further heteroatom selected from the group consisting of O, N and S as ring member and/or may carry one or more substituents selected from the group consisting of halogen, C₁-C₆-alkyl, C₁-C₆-haloalkyl, C₂-C₆-alkenyl, C₂-C₆-haloalkenyl, C₁-C₆-alkoxy, C₁-C₆-haloalkoxy, C₃-C₆-alkenyloxy, C₃-C₆-haloalkenyloxy, C₁-C₆-alkylene and oxy-C₁-C₃-alkyleneoxy. These compounds correspond in particular to formula I.2, 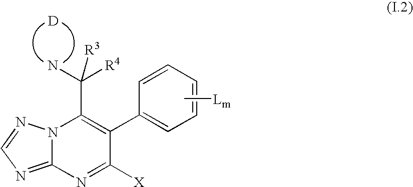
in which
D together with the nitrogen atom forms a five- or six-membered heterocyclyl or heteroaryl as defined above which is attached via N and which may contain a further heteroatom selected from the group consisting of O, N and S as ring member and/or may carry one or more substituents selected from the group consisting of halogen, C₁-C₄-alkyl, C₁-C₄-alkoxy and C₁-C₂-haloalkyl;
R³ and R⁴ independently of one another are hydrogen or C₁-C₄-alkyl;
X is chlorine, methyl, cyano, methoxy or ethoxy;
L is halogen, cyano, nitro, C₁-C₆-alkyl, C₁-C₆-haloalkyl, C₁-C₆-alkoxy, a group C(S)A² or a group C(O)A² in which A² is C₁-C₄-alkoxy, NH₂, C₁-C₄-alkylamino or di-C₁-C₄-alkylamino; and
m is 0, 1, 2, 3, 4 or 5.

A further preferred embodiment of the invention relates to compounds of the formula I.3. 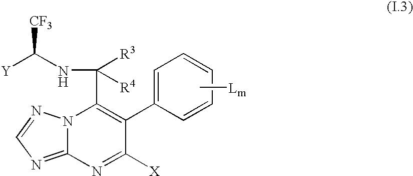
in which
Y is hydrogen or C₁-C₄-alkyl, in particular methyl and ethyl,
R³ and R⁴ independently of one another are hydrogen or C₁-C₄-alkyl;
X is chlorine, methyl, cyano, methoxy or ethoxy;
L is halogen, cyano, nitro, C₁-C₆-alkyl, C₁-C₆-haloalkyl, C₁-C₆-alkoxy, a group C(S)A² or a group C(O)A² in which A² is C₁-C₄-alkoxy, NH₂, C₁-C₄-alkylamino or di-C₁-C₄-alkylamino; and
m is 0, 1, 2, 3, 4 or 5.

In particular with a view to their use, preference is given to the compounds I compiled in tables 1 to 128 below. The groups mentioned for a substituent in tables 1 to 128 are furthermore per se, independently of the combination in which they are mentioned, a particularly preferred embodiment of the substituent in question.

Table 1

Compounds of the formula I in which X is chlorine, Lₘ is 2-fluoro-6-chloro, R³ and R⁴ are each hydrogen and the combination of R¹ and R² for a compound corresponds in each case to one row of Table A.

Table 2

Compounds of the formula I in which X is chlorine, Lₘ is 2,6-difluoro, R³ and R⁴ are each hydrogen and the combination of R¹ and R² for a compound corresponds in each case to one row of Table A.

Table 3

Compounds of the formula I in which X is chlorine, Lₘ is 2,6-dichloro, R³ and R⁴ are each hydrogen and the combination of R¹ and R² for a compound corresponds in each case to one row of Table A.

Table 4

Compounds of the formula I in which X is chlorine, Lₘ is 2-fluoro-6-methyl, R³ and R⁴ are each hydrogen and the combination of R¹ and R² for a compound corresponds in each case to one row of Table A.

Table 5

Compounds of the formula I in which X is chlorine, Lₘ is 2,4,6-trifluoro, R³ and R⁴ are each hydrogen and the combination of R¹ and R² for a compound corresponds in each case to one row of Table A.

Table 6

Compounds of the formula I in which X is chlorine, Lₘ is 2,6-difluoro-4-methoxy, R³ and R⁴ are each hydrogen and the combination of R¹ and R² for a compound corresponds in each case to one row of Table A.

Table 7

Compounds of the formula I in which X is chlorine, Lₘ is 2-fluoro-6-chloro, R³ and R⁴ are each hydrogen and the combination of R¹ and R² for a compound corresponds in each case to one row of Table A.

Table 8

Compounds of the formula I in which X is chlorine, Lₘ is pentafluoro, R³ and R⁴ are each hydrogen and the combination of R¹ and R² for a compound corresponds in each case to one row of Table A.

Table 9

Compounds of the formula I in which X is chlorine, Lₘ is 2-methyl-4-fluoro, R³ and R⁴ are each hydrogen and the combination of R¹ and R² for a compound corresponds in each case to one row of Table A.

Table 10

Compounds of the formula I in which X is chlorine, Lₘ is 2-trifluoromethyl, R³ and R⁴ are each hydrogen and the combination of R¹ and R² for a compound corresponds in each case to one row of Table A.

Table 11

Compounds of the formula I in which X is chlorine, Lₘ is 2-methoxy-6-fluorophenyl, R³ and R⁴ are each hydrogen and the combination of R¹ and R² for a compound corresponds in each case to one row of Table A.

Table 12

Compounds of the formula I in which X is chlorine, Lₘ is 2-chlorophenyl, R³ and R⁴ are each hydrogen and the combination of R¹ and R² for a compound corresponds in each case to one row of Table A.

Table 13

Compounds of the formula I in which X is chlorine, Lₘ is 2-fluorophenyl, R³ and R⁴ are each hydrogen and the combination of R¹ and R² for a compound corresponds in each case to one row of Table A.

Table 14

Compounds of the formula I in which X is chlorine, Lₘ is 2,4-difluorophenyl, R³ and R⁴ are each hydrogen and the combination of R¹ and R² for a compound corresponds in each case to one row of Table A.

Table 15

Compounds of the formula I in which X is chlorine, Lₘ is 2-fluoro-4-chloro, R³ and R⁴ are each hydrogen and the combination of R¹ and R² for a compound corresponds in each case to one row of Table A.

Table 16

Compounds of the formula I in which X is chlorine, Lₘ is 2-chloro-4-fluorophenyl, R³ and R⁴ are each hydrogen and the combination of R¹ and R² for a compound corresponds in each case to one row of Table A.

Table 17

Compounds of the formula I in which X is chlorine, Lₘ is 2,3-difluoro, R³ and R⁴ are each hydrogen and the combination of R¹ and R² for a compound corresponds in each case to one row of Table A.

Table 18

Compounds of the formula I in which X is chlorine, Lₘ is 2,5-difluoro, R³ and R⁴ are each hydrogen and the combination of R¹ and R² for a compound corresponds in each case to one row of Table A.

Table 19

Compounds of the formula I in which X is chlorine, Lₘ is 2-methyl-5-fluoro, R³ and R⁴ are each hydrogen and the combination of R¹ and R² for a compound corresponds in each case to one row of Table A.

Table 20

Compounds of the formula I in which X is chlorine, Lₘ is 2,3,4-trifluorophenyl, R³ and R⁴ are each hydrogen and the combination of R¹ and R² for a compound corresponds in each case to one row of Table A.

Table 21

Compounds of the formula I in which X is chlorine, Lₘ is 2-methyl, R³ and R⁴ are each hydrogen and the combination of R¹ and R² for a compound corresponds in each case to one row of Table A.

Table 22

Compounds of the formula I in which X is chlorine, Lₘ is 2,4-dimethyl, R³ and R⁴ are each hydrogen and the combination of R¹ and R² for a compound corresponds in each case to one row of Table A.

Table 23

Compounds of the formula I in which X is chlorine, Lₘ is 2-methyl-4-chloro, R³ and R⁴ are each hydrogen and the combination of R¹ and R² for a compound corresponds in each case to one row of Table A.

Table 24

Compounds of the formula I in which X is chlorine, Lₘ is 2-fluoro-4-methyl, R³ and R⁴ are each hydrogen and the combination of R¹ and R² for a compound corresponds in each case to one row of Table A.

Table 25

Compounds of the formula I in which X is chlorine, Lₘ is 2,6-dimethyl, R³ and R⁴ are each hydrogen and the combination of R¹ and R² for a compound corresponds in each case to one row of Table A.

Table 26

Compounds of the formula I in which X is chlorine, Lₘ is 2,4,5-trimethylphenyl, R³ and

R⁴ are each hydrogen and the combination of R¹ and R² for a compound corresponds in each case to one row of Table A.

Table 27

Compounds of the formula I in which X is chlorine, Lₘ is 2,6-difluoro-4-cyano, R³ and

R⁴ are each hydrogen and the combination of R¹ and R² for a compound corresponds in each case to one row of Table A.

Table 28

Compounds of the formula I in which X is chlorine, Lₘ is 2,6-difluoro-4-methyl, R³ and

R⁴ are each hydrogen and the combination of R¹ and R² for a compound corresponds in each case to one row of Table A.

Table 29

Compounds of the formula I in which X is chlorine, Lₘ is 2,6-difluoro-4-methoxycarbonyl, R³ and R⁴ are each hydrogen and the combination of R¹ and R² for a compound corresponds in each case to one row of Table A.

Table 30

Compounds of the formula I in which X is chlorine, Lₘ is 2-trifluoromethyl-4-fluoro, R³ and R⁴ are each hydrogen and the combination of R¹ and R² for a compound corresponds in each case to one row of Table A.

Table 31

Compounds of the formula I in which X is chlorine, Lₘ is 2-trifluoromethyl-5-fluoro, R³ and R⁴ are each hydrogen and the combination of R¹ and R² for a compound corresponds in each case to one row of Table A.

Table 32

Compounds of the formula I in which X is chlorine, Lₘ is 2-trifluoromethyl-5-chloro, R³ and R⁴ are each hydrogen and the combination of R¹ and R² for a compound corresponds in each case to one row of Table A.

Table 33

Compounds of the formula I in which X is methyl, Lₘ is 2-fluoro-6-chloro, R³ and R⁴ are each hydrogen and the combination of R¹ and R² for a compound corresponds in each case to one row of Table A.

Table 34

Compounds of the formula I in which X is methyl, Lₘ is 2,6-difluoro, R³ and R⁴ are each hydrogen and the combination of R¹ and R² for a compound corresponds in each case to one row of Table A.

Table 35

Compounds of the formula I in which X is methyl, Lₘ is 2,6-dichloro, R³ and R⁴ are each hydrogen and the combination of R¹ and R² for a compound corresponds in each case to one row of Table A.

Table 36

Compounds of the formula I in which X is methyl, Lₘ is 2-fluoro-6-methyl, R³ and R⁴ are each hydrogen and the combination of R¹ and R² for a compound corresponds in each case to one row of Table A.

Table 37

Compounds of the formula I in which X is methyl, Lₘ is 2,4,6-trifluoro, R³ and R⁴ are each hydrogen and the combination of R¹ and R² for a compound corresponds in each case to one row of Table A.

Table 38

Compounds of the formula I in which X is methyl, Lₘ is 2,6-difluoro-4-methoxy, R³ and R⁴ are each hydrogen and the combination of R¹ and R² for a compound corresponds in each case to one row of Table A.

Table 39

Compounds of the formula I in which X is methyl, Lₘ is 2-fluoro-6-chloro, R³ and R⁴ are each hydrogen and the combination of R¹ and R² for a compound corresponds in each case to one row of Table A.

Table 40

Compounds of the formula I in which X is methyl, Lₘ is pentafluoro, R³ and R⁴ are each hydrogen and the combination of R¹ and R² for a compound corresponds in each case to one row of Table A.

Table 41

Compounds of the formula I in which X is methyl, Lₘ is 2-methyl-4-fluoro, R³ and R⁴ are each hydrogen and the combination of R¹ and R² for a compound corresponds in each case to one row of Table A.

Table 42

Compounds of the formula I in which X is methyl, Lₘ is 2-trifluoromethyl, R³ and R⁴ are each hydrogen and the combination of R¹ and R² for a compound corresponds in each case to one row of Table A.

Table 43

Compounds of the formula I in which X is methyl, Lₘ is 2-methoxy-6-fluorophenyl, R³ and R⁴ are each hydrogen and the combination of R¹ and R² for a compound corresponds in each case to one row of Table A.

Table 44

Compounds of the formula I in which X is methyl, Lₘ is 2-chlorophenyl, R³ and R⁴ are each hydrogen and the combination of R¹ and R² for a compound corresponds in each case to one row of Table A.

Table 45

Compounds of the formula I in which X is methyl, Lₘ is 2-fluorophenyl, R³ and R⁴ are each hydrogen and the combination of R¹ and R² for a compound corresponds in each case to one row of Table A.

Table 46

Compounds of the formula I in which X is methyl, Lₘ is 2,4-difluorophenyl, R³ and R⁴ are each hydrogen and the combination of R¹ and R² for a compound corresponds in each case to one row of Table A.

Table 47

Compounds of the formula I in which X is methyl, Lₘ is 2-fluoro-4-chloro, R³ and R⁴ are each hydrogen and the combination of R¹ and R² for a compound corresponds in each case to one row of Table A.

Table 48

Compounds of the formula I in which X is methyl, Lₘ is 2-chloro-4-fluorophenyl, R³ and R⁴ are each hydrogen and the combination of R¹ and R² for a compound corresponds in each case to one row of Table A.

Table 49

Compounds of the formula I in which X is methyl, Lₘ is 2,3-difluoro, R³ and R⁴ are each hydrogen and the combination of R¹ and R² for a compound corresponds in each case to one row of Table A.

Table 50

Compounds of the formula I in which X is methyl, Lₘ is 2,5-difluoro, R³ and R⁴ are each hydrogen and the combination of R¹ and R² for a compound corresponds in each case to one row of Table A.

Table 51

Compounds of the formula I in which X is methyl, Lₘ is 2-methyl-5-fluoro, R³ and R⁴ are each hydrogen and the combination of R¹ and R² for a compound corresponds in each case to one row of Table A.

Table 52

Compounds of the formula I in which X is methyl, Lₘ is 2,3,4-trifluorophenyl, R³ and R⁴ are each hydrogen and the combination of R¹ and R² for a compound corresponds in each case to one row of Table A.

Table 53

Compounds of the formula I in which X is methyl, Lₘ is 2-methyl, R³ and R⁴ are each hydrogen and the combination of R¹ and R² for a compound corresponds in each case to one row of Table A.

Table 54

Compounds of the formula I in which X is methyl, Lₘ is 2,4-dimethyl, R³ and R⁴ are each hydrogen and the combination of R¹ and R² for a compound corresponds in each case to one row of Table A.

Table 55

Compounds of the formula I in which X is methyl, Lₘ is 2-methyl-4-chloro, R³ and R⁴ are each hydrogen and the combination of R¹ and R² for a compound corresponds in each case to one row of Table A.

Table 56

Compounds of the formula I in which X is methyl, Lₘ is 2-fluoro-4-methyl, R³ and R⁴ are each hydrogen and the combination of R¹ and R² for a compound corresponds in each case to one row of Table A.

Table 57

Compounds of the formula I in which X is methyl, Lₘ is 2,6-dimethyl, R³ and R⁴ are each hydrogen and the combination of R¹ and R² for a compound corresponds in each case to one row of Table A.

Table 58

Compounds of the formula I in which X is methyl, Lₘ is 2,4,5-trimethylphenyl, R³ and R⁴ are each hydrogen and the combination of R¹ and R² for a compound corresponds in each case to one row of Table A.

Table 59

Compounds of the formula I in which X is methyl, Lₘ is 2,6-difluoro-4-cyano, R³ and R⁴ are each hydrogen and the combination of R¹ and R² for a compound corresponds in each case to one row of Table A.

Table 60

Compounds of the formula I in which X is methyl, Lₘ is 2,6-difluoro-4-methyl, R³ and R⁴ are each hydrogen and the combination of R¹ and R² for a compound corresponds in each case to one row of Table A.

Table 61

Compounds of the formula I in which X is methyl, Lₘ is 2,6-difluoro-4-methoxycarbonyl, R³ and R⁴ are each hydrogen and the combination of R¹ and R² for a compound corresponds in each case to one row of Table A.

Table 62

Compounds of the formula I in which X is methyl, Lₘ is 2-trifluoromethyl-4-fluoro, R³ and R⁴ are each hydrogen and the combination of R¹ and R² for a compound corresponds in each case to one row of Table A.

Table 63

Compounds of the formula I in which X is methyl, Lₘ is 2-trifluoromethyl-5-fluoro, R³ and R⁴ are each hydrogen and the combination of R¹ and R² for a compound corresponds in each case to one row of Table A.

Table 64

Compounds of the formula I in which X is methyl, Lₘ is 2-trifluoromethyl-5-chloro, R³ and R⁴ are each hydrogen and the combination of R¹ and R² for a compound corresponds in each case to one row of Table A.

Table 65

Compounds of the formula I in which X is methoxy, Lₘ is 2-fluoro-6-chloro, R³ and R⁴ are each hydrogen and the combination of R¹ and R² for a compound corresponds in each case to one row of Table A.

Table 66

Compounds of the formula I in which X is methoxy, Lₘ is 2,6-difluoro, R³ and R⁴ are each hydrogen and the combination of R¹ and R² for a compound corresponds in each case to one row of Table A.

Table 67

Compounds of the formula I in which X is methoxy, Lₘ is 2,6-dichloro, R³ and R⁴ are each hydrogen and the combination of R¹ and R² for a compound corresponds in each case to one row of Table A.

Table 68

Compounds of the formula I in which X is methoxy, Lₘ is 2-fluoro-6-methyl, R³ and R⁴ are each hydrogen and the combination of R¹ and R² for a compound corresponds in each case to one row of Table A.

Table 69

Compounds of the formula I in which X is methoxy, Lₘ is 2,4,6-trifluoro, R³ and R⁴ are each hydrogen and the combination of R¹ and R² for a compound corresponds in each case to one row of Table A.

Table 70

Compounds of the formula I in which X is methoxy, Lₘ is 2,6-difluoro-4-methoxy, R³ and R⁴ are each hydrogen and the combination of R¹ and R² for a compound corresponds in each case to one row of Table A.

Table 71

Compounds of the formula I in which X is methoxy, Lₘ is 2-fluoro-6-chloro, R³ and R⁴ are each hydrogen and the combination of R¹ and R² for a compound corresponds in each case to one row of Table A.

Table 72

Compounds of the formula I in which X is methoxy, Lₘ is pentafluoro, R³ and R⁴ are each hydrogen and the combination of R¹ and R² for a compound corresponds in each case to one row of Table A.

Table 73

Compounds of the formula I in which X is methoxy, Lₘ is 2-methyl-4-fluoro, R³ and R⁴ are each hydrogen and the combination of R¹ and R² for a compound corresponds in each case to one row of Table A.

Table 74

Compounds of the formula I in which X is methoxy, Lₘ is 2-trifluoromethyl, R³ and R⁴ are each hydrogen and the combination of R¹ and R² for a compound corresponds in each case to one row of Table A.

Table 75

Compounds of the formula I in which X is methoxy, Lₘ is 2-methoxy-6-fluorophenyl, R³ and R⁴ are each hydrogen and the combination of R¹ and R² for a compound corresponds in each case to one row of Table A.

Table 76

Compounds of the formula I in which X is methoxy, Lₘ is 2-chlorophenyl, R³ and R⁴ are each hydrogen and the combination of R¹ and R² for a compound corresponds in each case to one row of Table A.

Table 77

Compounds of the formula I in which X is methoxy, Lₘ is 2-fluorophenyl, R³ and R⁴ are each hydrogen and the combination of R¹ and R² for a compound corresponds in each case to one row of Table A.

Table 78

Compounds of the formula I in which X is methoxy, Lₘ is 2,4-difluorophenyl, R³ and R⁴ are each hydrogen and the combination of R¹ and R² for a compound corresponds in each case to one row of Table A.

Table 79

Compounds of the formula I in which X is methoxy, Lₘ is 2-fluoro-4-chloro, R³ and R⁴ are each hydrogen and the combination of R¹ and R² for a compound corresponds in each case to one row of Table A.

Table 80

Compounds of the formula I in which X is methoxy, Lₘ is 2-chloro-4-fluorophenyl, R³ and R⁴ are each hydrogen and the combination of R¹ and R² for a compound corresponds in each case to one row of Table A.

Table 81

Compounds of the formula I in which X is methoxy, Lₘ is 2,3-difluoro, R³ and R⁴ are each hydrogen and the combination of R¹ and R² for a compound corresponds in each case to one row of Table A.

Table 82

Compounds of the formula I in which X is methoxy, Lₘ is 2,5-difluoro, R³ and R⁴ are each hydrogen and the combination of R¹ and R² for a compound corresponds in each case to one row of Table A.

Table 83

Compounds of the formula I in which X is methoxy, Lₘ is 2-methyl-5-fluoro, R³ and R⁴ are each hydrogen and the combination of R¹ and R² for a compound corresponds in each case to one row of Table A.

Table 84

Compounds of the formula I in which X is methoxy, Lₘ is 2,3,4-trifluorophenyl, R³ and R⁴ are each hydrogen and the combination of R¹ and R² for a compound corresponds in each case to one row of Table A.

Table 85

Compounds of the formula I in which X is methoxy, Lₘ is 2-methyl, R³ and R⁴ are each hydrogen and the combination of R¹ and R² for a compound corresponds in each case to one row of Table A.

Table 86

Compounds of the formula I in which X is methoxy, Lₘ is 2,4-dimethyl, R³ and R⁴ are each hydrogen and the combination of R¹ and R² for a compound corresponds in each case to one row of Table A.

Table 87

Compounds of the formula I in which X is methoxy, Lₘ is 2-methyl-4-chloro, R³ and R⁴ are each hydrogen and the combination of R¹ and R² for a compound corresponds in each case to one row of Table A.

Table 88

Compounds of the formula I in which X is methoxy, Lₘ is 2-fluoro-4-methyl, R³ and R⁴ are each hydrogen and the combination of R¹ and R² for a compound corresponds in each case to one row of Table A.

Table 89

Compounds of the formula I in which X is methoxy, Lₘ is 2,6-dimethyl, R³ and R⁴ are each hydrogen and the combination of R¹ and R² for a compound corresponds in each case to one row of Table A.

Table 90

Compounds of the formula I in which X is methoxy, Lₘ is 2,4,5-trimethylphenyl, R³ and R⁴ are each hydrogen and the combination of R¹ and R² for a compound corresponds in each case to one row of Table A.

Table 91

Compounds of the formula I in which X is methoxy, Lₘ is 2,6-difluoro-4-cyano, R³ and R⁴ are each hydrogen and the combination of R¹ and R² for a compound corresponds in each case to one row of Table A.

Table 92

Compounds of the formula I in which X is methoxy, Lₘ is 2,6-difluoro-4-methyl, R³ and R⁴ are each hydrogen and the combination of R¹ and R² for a compound corresponds in each case to one row of Table A.

Table 93

Compounds of the formula I in which X is methoxy, Lₘ is 2,6-difluoro-4-methoxycarbonyl, R³ and R⁴ are each hydrogen and the combination of R¹ and R² for a compound corresponds in each case to one row of Table A.

Table 94

Compounds of the formula I in which X is methoxy, Lₘ is 2-trifluoromethyl-4-fluoro, R³ and R⁴ are each hydrogen and the combination of R¹ and R² for a compound corresponds in each case to one row of Table A.

Table 95

Compounds of the formula I in which X is methoxy, Lₘ is 2-trifluoromethyl-5-fluoro, R³ and R⁴ are each hydrogen and the combination of R¹ and R² for a compound corresponds in each case to one row of Table A.

Table 96

Compounds of the formula I in which X is methoxy, Lₘ is 2-trifluoromethyl-5-chloro, R³ and R⁴ are each hydrogen and the combination of R¹ and R² for a compound corresponds in each case to one row of Table A.

Table 97

Compounds of the formula I in which X is cyano, Lₘ is 2-fluoro-6-chloro, R³ and R⁴ are each hydrogen and the combination of R¹ and R² for a compound corresponds in each case to one row of Table A.

Table 98

Compounds of the formula I in which X is cyano, Lₘ is 2,6-difluoro, R³ and R⁴ are each hydrogen and the combination of R¹ and R² for a compound corresponds in each case to one row of Table A.

Table 99

Compounds of the formula I in which X is cyano, Lₘ is 2,6-dichloro, R³ and R⁴ are each hydrogen and the combination of R¹ and R² for a compound corresponds in each case to one row of Table A.

Table 100

Compounds of the formula I in which X is cyano, Lₘ is 2-fluoro-6-methyl, R³ and R⁴ are each hydrogen and the combination of R¹ and R² for a compound corresponds in each case to one row of Table A.

Table 101

Compounds of the formula I in which X is cyano, Lₘ is 2,4,6-trifluoro, R³ and R⁴ are each hydrogen and the combination of R¹ and R² for a compound corresponds in each case to one row of Table A.

Table 102

Compounds of the formula I in which X is cyano, Lₘ is 2,6-difluoro-4-methoxy, R³ and R⁴ are each hydrogen and the combination of R¹ and R² for a compound corresponds in each case to one row of Table A.

Table 103

Compounds of the formula I in which X is cyano, Lₘ is 2-fluoro-6-chloro, R³ and R⁴ are each hydrogen and the combination of R¹ and R² for a compound corresponds in each case to one row of Table A.

Table 104

Compounds of the formula I in which X is cyano, Lₘ is pentafluoro, R³ and R⁴ are each hydrogen and the combination of R¹ and R² for a compound corresponds in each case to one row of Table A.

Table 105

Compounds of the formula I in which X is cyano, Lₘ is 2-methyl-4-fluoro, R³ and R⁴ are each hydrogen and the combination of R¹ and R² for a compound corresponds in each case to one row of Table A.

Table 106

Compounds of the formula I in which X is cyano, Lₘ is 2-trifluoromethyl, R³ and R⁴ are each hydrogen and the combination of R¹ and R² for a compound corresponds in each case to one row of Table A.

Table 107

Compounds of the formula I in which X is cyano, Lₘ is 2-methoxy-6-fluorophenyl, R³ and R⁴ are each hydrogen and the combination of R¹ and R² for a compound corresponds in each case to one row of Table A.

Table 108

Compounds of the formula I in which X is cyano, Lₘ is 2-chlorophenyl, R³ and R⁴ are each hydrogen and the combination of R¹ and R² for a compound corresponds in each case to one row of Table A.

Table 109

Compounds of the formula I in which X is cyano, Lₘ is 2-fluorophenyl, R³ and R⁴ are each hydrogen and the combination of R¹ and R² for a compound corresponds in each case to one row of Table A.

Table 110

Compounds of the formula I in which X is cyano, Lₘ is 2,4-difluorophenyl, R³ and R⁴ are each hydrogen and the combination of R¹ and R² for a compound corresponds in each case to one row of Table A.

Table 111

Compounds of the formula I in which X is cyano, Lₘ is 2-fluoro-4-chloro, R³ and R⁴ are each hydrogen and the combination of R¹ and R² for a compound corresponds in each case to one row of Table A.

Table 112

Compounds of the formula I in which X is cyano, Lₘ is 2-chloro-4-fluorophenyl, R³ and R⁴ are each hydrogen and the combination of R¹ and R² for a compound corresponds in each case to one row of Table A.

Table 113

Compounds of the formula I in which X is cyano, Lₘ is 2,3-difluoro, R³ and R⁴ are each hydrogen and the combination of R¹ and R² for a compound corresponds in each case to one row of Table A.

Table 114

Compounds of the formula I in which X is cyano, Lₘ is 2,5-difluoro, R³ and R⁴ are each hydrogen and the combination of R¹ and R² for a compound corresponds in each case to one row of Table A.

Table 115

Compounds of the formula I in which X is cyano, Lₘ is 2-methyl-5-fluoro, R³ and R⁴ are each hydrogen and the combination of R¹ and R² for a compound corresponds in each case to one row of Table A.

Table 116

Compounds of the formula I in which X is cyano, Lₘ is 2,3,4-trifluorophenyl, R³ and R⁴ are each hydrogen and the combination of R¹ and R² for a compound corresponds in each case to one row of Table A.

Table 117

Compounds of the formula I in which X is cyano, Lₘ is 2-methyl, R³ and R⁴ are each hydrogen and the combination of R¹ and R² for a compound corresponds in each case to one row of Table A.

Table 118

Compounds of the formula I in which X is cyano, Lₘ is 2,4-dimethyl, R³ and R⁴ are each hydrogen and the combination of R¹ and R² for a compound corresponds in each case to one row of Table A.

Table 119

Compounds of the formula I in which X is cyano, Lₘ is 2-methyl-4-chloro, R³ and R⁴ are each hydrogen and the combination of R¹ and R² for a compound corresponds in each case to one row of Table A.

Table 120

Compounds of the formula I in which X is cyano, Lₘ is 2-fluoro-4-methyl, R³ and R⁴ are each hydrogen and the combination of R¹ and R² for a compound corresponds in each case to one row of Table A.

Table 121

Compounds of the formula I in which X is cyano, Lₘ is 2,6-dimethyl, R³ and R⁴ are each hydrogen and the combination of R¹ and R² for a compound corresponds in each case to one row of Table A.

Table 122

Compounds of the formula I in which X is cyano, Lₘ is 2,4,5-trimethylphenyl, R³ and R⁴ are each hydrogen and the combination of R¹ and R² for a compound corresponds in each case to one row of Table A.

Table 123

Compounds of the formula I in which X is cyano, Lₘ is 2,6-difluoro-4-cyano, R³ and R⁴ are each hydrogen and the combination of R¹ and R² for a compound corresponds in each case to one row of Table A.

Table 124

Compounds of the formula I in which X is cyano, Lₘ is 2,6-difluoro-4-methyl, R³ and R⁴ are each hydrogen and the combination of R¹ and R² for a compound corresponds in each case to one row of Table A.

Table 125

Compounds of the formula I in which X is cyano, Lₘ is 2,6-difluoro-4-methoxycarbonyl, R³ and R⁴ are each hydrogen and the combination of R¹ and R² for a compound corresponds in each case to one row of Table A.

Table 126

Compounds of the formula I in which X is cyano, Lₘ is 2-trifluoromethyl-4-fluoro, R³ and R⁴ are each hydrogen and the combination of R¹ and R² for a compound corresponds in each case to one row of Table A.

Table 127

Compounds of the formula I in which X is cyano, Lₘ is 2-trifluoromethyl-5-fluoro, R³ and R⁴ are each hydrogen and the combination of R¹ and R² for a compound corresponds in each case to one row of Table A.

Table 128

Compounds of the formula I in which X is cyano, Lₘ is 2-trifluoromethyl-5-chloro, R³ and R⁴ are each hydrogen and the combination of R¹ and R² for a compound corresponds in each case to one row of Table A.

TABLE A

| No. | R¹ | R² |
|---|---|---|
| A-1 | H | H |
| A-2 | CH₃ | H |
| A-3 | CH₃ | CH₃ |
| A-4 | CH₂CH₃ | H |
| A-5 | CH₂CH₃ | CH₃ |
| A-6 | CH₂CH₃ | CH₂CH₃ |
| A-7 | CH₂CF₃ | H |
| A-8 | CH₂CF₃ | CH₃ |
| A-9 | CH₂CF₃ | CH₂CH₃ |
| A-10 | CH₂CCl₃ | H |
| A-11 | CH₂CCl₃ | CH₃ |
| A-12 | CH₂CCl₃ | CH₂CH₃ |
| A-13 | CH₂CH₂CH₃ | H |
| A-14 | CH₂CH₂CH₃ | CH₃ |
| A-15 | CH₂CH₂CH₃ | CH₂CH₃ |
| A-16 | CH₂CH₂CH₃ | CH₂CH₂CH₃ |
| A-17 | CH(CH₃)₂ | H |
| A-18 | CH(CH₃)₂ | CH₃ |
| A-19 | CH(CH₃)₂ | CH₂CH₃ |
| A-20 | CH₂CH₂CH₂CH₃ | H |
| A-21 | CH₂CH₂CH₂CH₃ | CH₃ |
| A-22 | CH₂CH₂CH₂CH₃ | CH₂CH₃ |
| A-23 | CH₂CH₂CH₂CH₃ | CH₂CH₂CH₃ |
| A-24 | CH₂CH₂CH₂CH₃ | CH₂CH₂CH₂CH₃ |
| A-25 | (±) CH(CH₃)—CH₂CH₃ | H |
| A-26 | (±) CH(CH₃)—CH₂CH₃ | CH₃ |
| A-27 | (±) CH(CH₃)—CH₂CH₃ | CH₂CH₃ |
| A-28 | (S) CH(CH₃)—CH₂CH₃ | H |
| A-29 | (S) CH(CH₃)—CH₂CH₃ | CH₃ |
| A-30 | (S) CH(CH₃)—CH₂CH₃ | CH₂CH₃ |
| A-31 | (R) CH(CH₃)—CH₂CH₃ | H |
| A-32 | (R) CH(CH₃)—CH₂CH₃ | CH₃ |
| A-33 | (R) CH(CH₃)—CH₂CH₃ | CH₂CH₃ |
| A-34 | (±) CH(CH₃)—CH(CH₃)₂ | H |
| A-35 | (±) CH(CH₃)—CH(CH₃)₂ | CH₃ |
| A-36 | (±) CH(CH₃)—CH(CH₃)₂ | CH₂CH₃ |
| A-37 | (S) CH(CH₃)—CH(CH₃)₂ | H |
| A-38 | (S) CH(CH₃)—CH(CH₃)₂ | CH₃ |
| A-39 | (S) CH(CH₃)—CH(CH₃)₂ | CH₂CH₃ |
| A-40 | (R) CH(CH₃)—CH(CH₃)₂ | H |
| A-41 | (R) CH(CH₃)—CH(CH₃)₂ | CH₃ |
| A-42 | (R) CH(CH₃)—CH(CH₃)₂ | CH₂CH₃ |
| A-43 | (±) CH(CH₃)—C(CH₃)₃ | H |
| A-44 | (±) CH(CH₃)—C(CH₃)₃ | CH₃ |
| A-45 | (±) CH(CH₃)—C(CH₃)₃ | CH₂CH₃ |
| A-46 | (S) CH(CH₃)—C(CH₃)₃ | H |
| A-47 | (S) CH(CH₃)—C(CH₃)₃ | CH₃ |
| A-48 | (S) CH(CH₃)—C(CH₃)₃ | CH₂CH₃ |
| A-49 | (R) CH(CH₃)—C(CH₃)₃ | H |
| A-50 | (R) CH(CH₃)—C(CH₃)₃ | CH₃ |
| A-51 | (R) CH(CH₃)—C(CH₃)₃ | CH₂CH₃ |
| A-52 | (±) CH(CH₃)—CF₃ | H |
| A-53 | (±) CH(CH₃)—CF₃ | CH₃ |
| A-54 | (±) CH(CH₃)—CF₃ | CH₂CH₃ |
| A-55 | (S) CH(CH₃)—CF₃ | H |
| A-56 | (S) CH(CH₃)—CF₃ | CH₃ |
| A-57 | (S) CH(CH₃)—CF₃ | CH₂CH₃ |
| A-58 | (R) CH(CH₃)—CF₃ | H |
| A-59 | (R) CH(CH₃)—CF₃ | CH₃ |
| A-60 | (R) CH(CH₃)—CF₃ | CH₂CH₃ |
| A-61 | (±) CH(CH₃)—CCl₃ | H |
| A-62 | (±) CH(CH₃)—CCl₃ | CH₃ |
| A-63 | (±) CH(CH₃)—CCl₃ | CH₂CH₃ |
| A-64 | (S) CH(CH₃)—CCl₃ | H |
| A-65 | (S) CH(CH₃)—CCl₃ | CH₃ |
| A-66 | (S) CH(CH₃)—CCl₃ | CH₂CH₃ |
| A-67 | (R) CH(CH₃)—CCl₃ | H |
| A-68 | (R) CH(CH₃)—CCl₃ | CH₃ |
| A-69 | (R) CH(CH₃)—CCl₃ | CH₂CH₃ |
| A-70 | CH₂CF₂CF₃ | H |
| A-71 | CH₂CF₂CF₃ | CH₃ |
| A-72 | CH₂CF₂CF₃ | CH₂CH₃ |
| A-73 | CH₂(CF₂)₂CF₃ | H |
| A-74 | CH₂(CF₂)₂CF₃ | CH₃ |
| A-75 | CH₂(CF₂)₂CF₃ | CH₂CH₃ |
| A-76 | CH₂C(CH₃)═CH₂ | H |
| A-77 | CH₂C(CH₃)═CH₂ | CH₃ |
| A-78 | CH₂C(CH₃)═CH₂ | CH₂CH₃ |
| A-79 | CH₂CH═CH₂ | H |
| A-80 | CH₂CH═CH₂ | CH₃ |
| A-81 | CH₂CH═CH₂ | CH₂CH₃ |
| A-82 | CH(CH₃)CH═CH₂ | H |
| A-83 | CH(CH₃)CH═CH₂ | CH₃ |
| A-84 | CH(CH₃)CH═CH₂ | CH₂CH₃ |
| A-85 | CH(CH₃)C(CH₃)═CH₂ | H |
| A-86 | CH(CH₃)C(CH₃)═CH₂ | CH₃ |
| A-87 | CH(CH₃)C(CH₃)═CH₂ | CH₂CH₃ |
| A-88 | CH₂—C≡CH | H |
| A-89 | CH₂—C≡CH | CH₃ |
| A-90 | CH₂—C≡CH | CH₂CH₃ |
| A-91 | cyclopentyl | H |
| A-92 | cyclopentyl | CH₃ |
| A-93 | cyclopentyl | CH₂CH₃ |
| A-94 | cyclohexyl | H |
| A-95 | cyclohexyl | CH₃ |
| A-96 | cyclohexyl | CH₂CH₃ |
| A-97 | CH₂—C₆H₅ | H |
| A-98 | CH₂—C₆H₅ | CH₃ |
| A-99 | CH₂—C₆H₅ | CH₂CH₃ |

| | |
|---|---|
| A-100 | —(CH₂)₂CH═CHCH₂— |
| A-101 | —(CH₂)₂C(CH₃)═CHCH₂— |
| A-102 | —CH(CH₃)CH₂—CH═CHCH₂— |
| A-103 | —(CH₂)₂CH(CH₃)(CH₂)₂— |
| A-104 | —(CH₂)₃CHFCH₂— |
| A-105 | —(CH₂)₂CHF(CH₂)₂— |
| A-106 | —CH₂CHF(CH₂)₃— |
| A-107 | —(CH₂)₂CH(CF₃)(CH₂)₂— |
| A-108 | —(CH₂)₂O(CH₂)₂— |
| A-109 | —(CH₂)₂S(CH₂)₂— |
| A-110 | —(CH₂)₅— |
| A-111 | —(CH₂)₄— |
| A-112 | —CH₂CH═CHCH₂— |
| A-113 | —CH(CH₃)(CH₂)₃— |
| A-114 | —CH₂CH(CH₃)(CH₂)₂— |
| A-115 | —CH(CH₃)—(CH₂)₂—CH(CH₃)— |
| A-116 | —CH(CH₃)—(CH₂)₄— |
| A-117 | —CH₂—CH(CH₃)—(CH₂)₃— |
| A-118 | —(CH₂)—CH(CH₃)—CH₂—CH(CH₃)—CH₂— |
| A-119 | —CH(CH₂CH₃)—(CH₂)₄— |
| A-120 | —(CH₂)₂—CHOH—(CH₂)₂— |
| A-121 | —(CH₂)₆— |
| A-122 | —CH(CH₃)—(CH₂)₅— |
| A-123 | —(CH₂)₂—N(CH₃)—(CH₂)₂— |
| A-124 | —N═CH—CH═CH— |
| A-125 | —N═C(CH₃)—CH═C(CH₃)— |
| A-126 | —N═C(CF₃)—CH═C(CF₃)— |

The compounds of the formula I according to the invention can be obtained by different routes analogously to prior art processes known per se, in accordance with the syntheses shown in the schemes below:

Compounds of the formula I in which X is Hal can be prepared, for example, as shown in scheme 1.

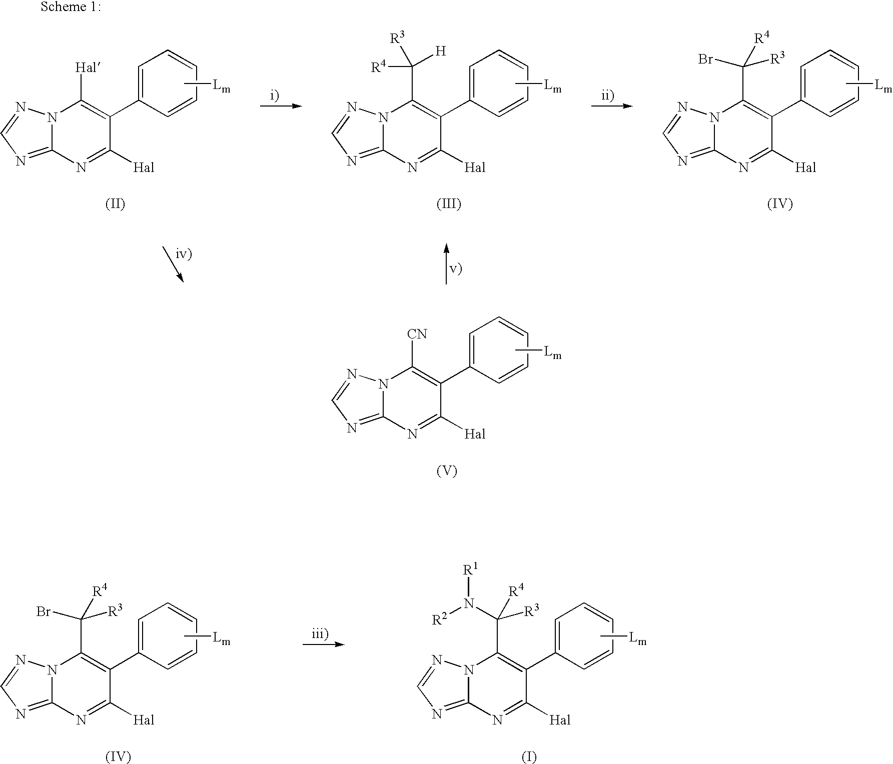

In scheme 1, R¹, R² and Lₘ have the meanings given above. R³ and R⁴ independently of one another are hydrogen or optionally substituted C₁-C₈-alkyl. Hal and Hal′ independently of one another are halogen, preferably chlorine or bromine.

In a first step i), for example, a 5,7-dihalotriazolopyrimidine of the formula II can be reacted with an organometallic compound of the formula VI
Met^{y}(CHR³R⁴)_{y} (VI)
in which Met is a metal atom or semimetal atom with the valency y, such as, for example, B, Zn, Mg, Ti or Sn, and R³ and R⁴ independently of one another are hydrogen or optionally substituted C₁-C₈-alkyl, which gives a 7-alkyl-6-aryltriazolopyrimidine compound of the formula II. In one embodiment of this process, the reaction is carried out with transition metal catalysis, such as Ni or Pd catalysis. This reaction can be carried out, for example, analogously to the following methods: J. Chem. Soc. Perkin Trans. 1 (1994), 1187, ibid. 1 (1996), 2345; WO-A 99/41255; Aust. J. Chem. 43 (1990), 733; J. Org. Chem., 43 (1978), 358; J. Chem. Soc. Chem. Commun. 866 (1979); Tetrahedron Lett., 34 (1993), 8267; ibid., 33 (1992), 413. The reaction can also be carried out in the absence of a catalyst, in particular when Met is Zn or Mg. The compounds II are known, for example, from U.S. Pat. No. 5,593,996, WO 94/20501, WO 98/46607 or WO 2004/041824.

The compounds of the formula II are known from the literature (WO99/41255 or U.S. Pat. No. 5,593,996) or can be prepared according to the literature cited.

The subsequent bromination of the 7-alkyl-6-aryltriazolopyrimidine compound of the formula III in step ii) affords the 6-aryl-7-(bromoalkyl)triazolopyrimidine compound of the formula IV. Suitable brominating agents are, for example, N-bromoimides, such as N-bromosuccinimide, N-bromohydantoin, bromine or pyridinium tribromide. Depending on the brominating agent, the addition of a free-radical initiator, for example an organic peroxide, such as dibenzoyl peroxide, or an azo compound, such as azobisisobutyronitrile, or irradiation with light may be advantageous for the course of the reaction. If a free-radical initiator is used, a catalytic amount is generally sufficient.

In general, the bromination is carried out in the presence of a solvent. Suitable solvents are, for example, aliphatic or aromatic hydrocarbons which may also be halogenated, for example carbon tetrachloride, trichloromethane, dichlormethane, organic acids, such as acetic acid, inorganic acids, pyridine, ethers, such as tetrahydrofuran or dioxane, sulfides, sulfoxides, sulfones and mixtures thereof.

The reaction temperature is generally between 10° C. and the boiling point of the solvent.

In a third step iii), the 7-(bromoalkyl)-6-aryltriazolopyrimidine compound of the formula IV is then reacted with an amine of the formula VII 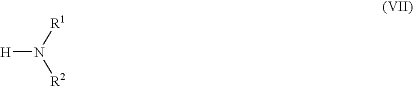
in which R¹ and R² have the meanings given above.

The reaction is advantageously carried out at from 0 to 70° C., preferably from 10 to 35° C. Preferably, the reaction is carried out in an inert solvent. Suitable solvents include ethers, such as dioxane, diethyl ether, methyl tert-butyl ether or, in particular, tetrahydrofuran, halogenated hydrocarbons, such as dichloromethane, and aromatic hydrocarbons, such as toluene, xylene and the like and mixtures thereof [cf. WO-A 98/46608].

The reaction can be carried out in the presence of a base, for example a tertiary amine, such as triethylamine, or an inorganic base, for example alkaline earth metal carbonates, alkaline earth metal bicarbonates, alkali metal carbonates or alkali metal bicarbonates, such as sodium carbonate, sodium bicarbonate, potassium carbonate or cesium carbonate; it is also possible for excess amine of the formula VII to serve as base.

The amines HNR¹R² are generally commercially available or can be prepared by known processes.

The compounds of the formula III can, for example, also be obtained advantageously by reacting compounds II with a cyanide (step iv in scheme 1) and subsequently reacting the 7-cyano-5-halotriazolopyrimdine V obtained with an organometallic reagent (step v in scheme 1).

In step iv), a 5,7-dihalotriazolopyrimidine II is initially reacted with a cyanide of the formula M-CN, giving a 7-cyano-5-halotriazolopyrimdine V. The cation M is of little importance; for practical reasons, preference is usually given to ammonium-, tetramethylammonium or tetraethylammonium cyanide, transition metal cyanides, such as zinc cyanide or alkali metal or alkaline earth metal cyanides. The reaction is preferably carried out in a solvent. Suitable solvents are ethers, such as diethyl ether, methyl tert-butyl ether, dioxane or tetrahydrofuran, aromatic hydrocarbons, such as toluene, carbonitriles, such as acetonitrile or propionitrile, and mixtures thereof. The reaction temperature is usually in the range from 0 to 120° C., preferably from 0 to 40° C.

The 7-cyano-5-halotriazolopyrimidine V obtained in this manner is then reacted with an organometallic compound, for example a Grignard compound of the formula (CHR³R⁴)—Mg-Hal or an organolithium compound of the formula (CHR³R⁴)—Li in which R³ and R⁴ independently of one another are hydrogen or optionally substituted C₁-C₈-alkyl and Hal is bromine or chlorine (step v). The reaction is usually carried out in an organic solvent. Suitable organic solvents are ethers, such as diethyl ether, dibutyl ether, methyl tert-butyl ether, tetrahydrofuran, aromatic hydrocarbons, such as toluene, and mixtures thereof. If appropriate, it may be advantageous to carry out the reaction in the presence of catalytic or, in particular, at least equimolar amounts of transition metal salts and/or compounds, in particular in the presence of Cu salts, such as Cu(I) halides, such as Cu(I) iodide. The reaction temperature is generally in a range of from −100 to +100° C., preferably below 0° C. and in particular in a range from −20 to −78° C.

Compounds of the formula I in which X is cyano, C₁-C₄-alkoxy or C₁-C₄-haloalkoxy can also be obtained advantageously by reacting compounds I in which X is halogen, preferably chlorine, with compounds M¹-X′ (formula VIII). Depending on the meaning of the group X′ to be introduced, the compounds VIII are inorganic cyanides, alkoxides or haloalkoxides. The reaction is advantageously carried out in the presence of an inert solvent. The cation M¹ in formula VIII is of little importance; for practical reasons, preference is usually given to ammonium salts, tetraalkylammonium salts, such as tetramethylammonium or tetraethylammonium salts, or alkali metal or alkaline earth metal salts (scheme 2).

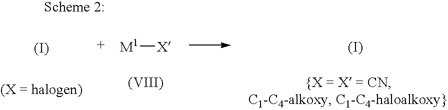

The reaction temperature is usually from 0 to 120° C., preferably from 10 to 40° C. [cf. J. Heterocycl. Chem., 12 (1975), 861-863].

Suitable solvents include ethers, such as dioxane, diethyl ether, methyl tert-butyl ether and, preferably, tetrahydrofuran, halogenated hydrocarbons, such as dichloromethane or dichloroethane, aromatic hydrocarbons, such as toluene, and mixtures thereof.

Compounds of the formula I in which X is C₁-C₄-alkyl, C₁-C₄-haloalkyl, C₂-C₈-alkenyl, C₂-C₆-haloalkenyl, C₂-C₈-alkynyl or C₂-C₈-haloalkynyl can be prepared in an advantageous manner by reacting compounds I in which X is halogen with organometallic compounds X^{a}—Mt in which X^{a} is C₁-C₄-alkyl, C₁-C₄-haloalkyl, C₂-C₈-alkenyl, C₂-C₈-haloalkenyl, C₂-C₈-alkynyl or C₂-C₈-haloalkynyl and Mt is lithium, magnesium or zinc. The reaction is preferably carried out in the presence of catalytic or in particular at least equimolar amounts of transiton metal salts and/or compounds, in particular in the presence of Cu salts such as Cu(I) halides and especially Cu(I) iodide. In general, the reaction is carried out in an inert organic solvent, for example one of the ethers mentioned above, in particular tetrahydrofuran, an aliphatic or cycloaliphatic hydrocarbon, such as hexane, cyclohexane and the like, an aromatic hydrocarbon, such as toluene, or in a mixture of these solvents. The required temperatures are in the range from −100 to +100° C. and especially in the range from −80° C. to +40° C. Methods to achieve this are known, for example from the prior art cited at the outset (see, for example, WO 03/004465).

Compounds of the formula I in which X is C₁-C₄-alkyl can in principle also be prepared by reacting compounds I in which X is halogen, in particular chlorine, with malonates of the formula IX. This synthesis route is shown in scheme 3. In formula IX, X″ is hydrogen or C₁-C₃-alkyl and R is C₁-C₄-alkyl. The compounds I are converted into compounds of the formula X which are then, after hydrolysis, decarboxylated to give compounds I [cf. U.S. Pat. No. 5,994,360].

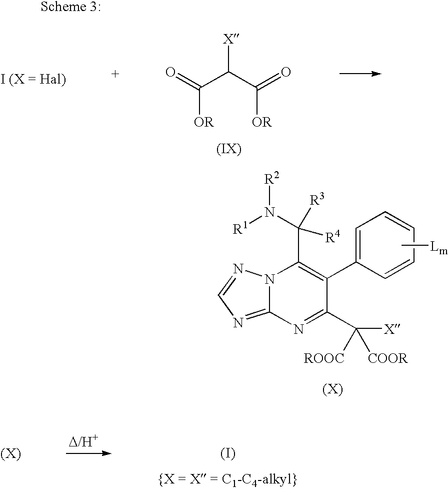

The malonates IX are known from the literature [J. Am. Chem. Soc. 64 (1942), 2714; J. Org. Chem. 39 (1974), 2172; Helv. Chim. Acta 61 (1978), 1565] or can be prepared in accordance with the literature cited.

The subsequent hydrolysis of the ester X is carried out under generally customary conditions. Depending on the various structural elements, alkaline or acidic hydrolysis of the compounds X may be advantageous. Under the conditions of ester hydrolysis, there may already be complete or partial decarboxylation to I.

The decarboxylation is usually carried out at temperatures of from 20° C. to 180° C., preferably from 50° C. to 120° C., in an inert solvent, if appropriate in the presence of an acid.

Suitable acids are hydrochloric acid, sulfuric acid, phosphoric acid, formic acid, acetic acid, p-toluenesulfonic acid. Suitable solvents are water, aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide; particularly preferably, the reaction is carried out in hydrochloric acid or acetic acid. It is also possible to use mixtures of the solvents mentioned.

A further synthesis route for compounds I in which X is C₁-C₄-alkyl is shown in scheme 4.

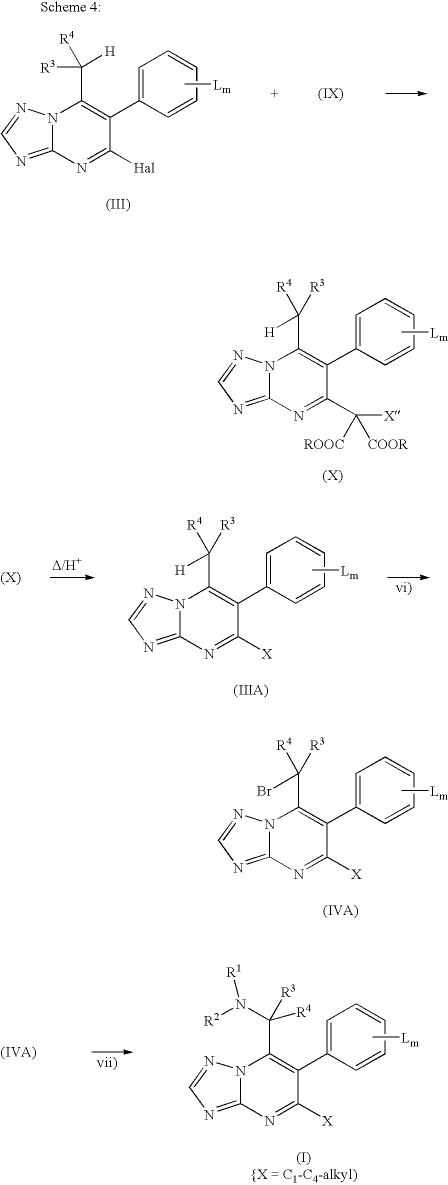

In scheme 4, Lₘ, R¹, R², R and X″ have the meanings given above. R³ and R⁴ independently of one another are hydrogen or optionally substituted C₁-C₈-alkyl. With respect to the conversion of the compound III where X=Hal into the compound IIIA where X=X″═C₁-C₄-alkyl, reference is made to what was said in scheme 2 with regard to the conversion of compounds of the formula I where X=Hal into compounds I where X=X″═C₁-C₄-alkyl in its entirety. Step vi in scheme 4 is carried out analogously to step ii) in scheme 1. Step vii) is carried out analogously to step iii) in scheme 1.

The reaction mixtures are worked up in a customary manner, for example by mixing with water, separating the phases and, if appropriate, chromatographic purification of the crude products. Some of the intermediates and end products are obtained in the form of colorless or slightly brownish viscous oils which are purified or freed from volatile components under reduced pressure and at moderately elevated temperature. If the intermediates and end products are obtained as solids, purification can also be carried out by recrystallization or digestion.

If individual compounds I cannot be obtained by the routes described above, they can be prepared by derivatization of other compounds I.

If the synthesis yields mixtures of isomers, a separation is generally however not necessarily required since in some cases the individual isomers can be interconverted during work-up for use or during application (for example under the action of light, acids or bases). Such conversions may also take place after use, for example, in the case of treatment of plants, in the treated plants, or in the harmful fungus to be controlled.

The compounds I are suitable as fungicides. They are distinguished by an outstanding effectiveness against a broad spectrum of phytopathogenic fungi, especially from the classes of the Ascomycetes, Deuteromycetes, Oomycetes and Basidiomycetes. Some are systemically effective and they can be used in plant protection as foliar and soil fungicides.

They are particularly important in the control of a multitude of fungi on various cultivated plants, such as wheat, rye, barley, oats, rice, corn, grass, bananas, cotton, soya, coffee, sugar cane, vines, fruits and ornamental plants, and vegetables, such as cucumbers, beans, tomatoes, potatoes and cucurbits, and on the seeds of these plants.

They are especially suitable for controlling the following plant diseases:
Alternaria species on fruit and vegetables,
Bipolaris and Drechslera species on cereals, rice and lawns,
Blumeria graminis (powdery mildew) on cereals,
Botrytis cinerea (gray mold) on strawberries, vegetables, ornamental plants and grapevines,
Erysiphe cichoracearum and Sphaerotheca fuliginea on cucurbits,
Fusarium and Verticillium species on various plants,
Mycosphaerella species on cereals, bananas and peanuts,
Phytophthora infestans on potatoes and tomatoes,
Plasmopara viticola on grapevines,
Podosphaera leucotricha on apples,
Pseudocercosporella herpotrichoides on wheat and barley,
Pseudoperonospora species on hops and cucumbers,
Puccinia species on cereals,
Pyricularia oryzae on rice,
Rhizoctonia species on cotton, rice and lawns,
Septoria tritici and Stagonospora nodorum on wheat,
Uncinula necator on grapevines,
Ustilago species on cereals and sugar cane, and
Venturia species (scab) on apples and pears.

The compounds I are also suitable for controlling harmful fungi, such as Paecilomyces variotii, in the protection of materials (e.g. wood, paper, paint dispersions, fibers or fabrics) and in the protection of stored products.

The compounds I are employed by treating the fungi or the plants, seeds, materials or soil to be protected from fungal attack with a fungicidally effective amount of the active compounds. The application can be carried out both before and after the infection of the materials, plants or seeds by the fungi.

The fungicidal compositions generally comprise between 0.1 and 95%, preferably between 0.5 and 90%, by weight of active compound.

When employed in plant protection, the amounts applied are, depending on the kind of effect desired, between 0.01 and 2.0 kg of active compound per ha.

In seed treatment, amounts of active compound of 1 to 1000 g, preferably 1 to 200 g, in particular 5 to 100 g per 100 kg of seed are generally used.

When used in the protection of materials or stored products, the amount of active compound applied depends on the kind of application area and on the desired effect. Amounts customarily applied in the protection of materials are, for example, 0.001 g to 2 kg, preferably 0.005 g to 1 kg, of active compound per cubic meter of treated material.

The compounds I can be converted into the customary formulations, for example solutions, emulsions, suspensions, dusts, powders, pastes and granules. The application form depends on the particular intended purpose; in each case, it should ensure a fine and uniform distribution of the compound according to the invention.

The formulations are prepared in a known manner, for example by extending the active compound with solvents and/or carriers, if desired using emulsifiers and dispersants. Solvents/auxiliaries which are suitable are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used,
carriers such as ground natural minerals (for example kaolins, clays, talc, chalk) and ground synthetic minerals (for example highly disperse silica, silicates); emulsifiers such as nonionic and anionic emulsifiers (for example polyoxyethylene fatty alcohol ethers, alkylsulfonates and arylsulfonates) and dispersants such as lignosulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ether, ethoxylated isooctylphenol, octylphenol, nonylphenol, alkylphenol polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound. The active compounds are employed in a purity of from 90% to 100%, preferably from 95% to 100% (according to NMR spectrum).

Examples of formulations include: 1. Products for dilution with water
A Water-soluble concentrates (SL)
   10 parts by weight of a compound according to the invention are dissolved in water or in a water-soluble solvent. As an alternative, wetters or other auxiliaries are added. The active compound dissolves upon dilution with water;
B Dispersible concentrates (DC)
   20 parts by weight of a compound according to the invention are dissolved in cyclohexanone with addition of a dispersant, for example polyvinylpyrrolidone. Dilution with water gives a dispersion;
C Emulsifiable concentrates (EC)
   15 parts by weight of a compound according to the invention are dissolved in xylene with addition of calcium dodecylbenzenesulfonate and castor oil ethoxylate (in each case 5%). Dilution with water gives an emulsion;
D Emulsions (EW, EO)
   40 parts by weight of a compound according to the invention are dissolved in xylene with addition of calcium dodecylbenzenesulfonate and castor oil ethoxylate (in each case 5%). This mixture is introduced into water by means of an emulsifying machine (Ultraturrax) and made into a homogeneous emulsion. Dilution with water gives an emulsion;
E Suspensions (SC, OD)
   In an agitated ball mill, 20 parts by weight of a compound according to the invention are comminuted with addition of dispersants, wetters and water or an organic solvent to give a fine active compound suspension. Dilution with water gives a stable suspension of the active compound;
F Water-dispersible granules and water-soluble granules (WG, SG)
   50 parts by weight of a compound according to the invention are ground finely with addition of dispersants and wetters and made into water-dispersible or water-soluble granules by means of technical appliances (for example extrusion, spray tower, fluidized bed). Dilution with water gives a stable dispersion or solution of the active compound;
G Water-dispersible powders and water-soluble powders (WP, SP)
   75 parts by weight of a compound according to the invention are ground in a rotor-stator mill with addition of dispersants, wetters and silica gel. Dilution with water gives a stable dispersion or solution of the active compound;
   2. Products to be applied undiluted, for example
H Dustable powders (DP)
   5 parts by weight of a compound according to the invention are ground finely and mixed intimately with 95% of finely divided kaolin. This gives a dustable product;
I Granules (GR, FG, GG, MG)
   0.5 part by weight of a compound according to the invention is ground finely and associated with 95.5% carriers. Current methods are extrusion, spray-drying or the fluidized bed. This gives granules to be applied undiluted;
J ULV solutions (UL)
   10 parts by weight of a compound according to the invention are dissolved in an organic solvent, for example xylene. This gives a product to be applied undiluted.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; the intention is to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), by which it is possible to apply formulations comprising over 95% by weight of active compound, or even to apply the active compound without additives.

Various types of oils, wetters, adjuvants, herbicides, fungicides, other pesticides, or bactericides may be added to the active compounds, if appropriate not until immediately prior to use (tank mix). These agents can be admixed with the agents according to the invention in a weight ratio of 1:10 to 10:1.

The compositions according to the invention can, in the use form as fungicides, also be present together with other active compounds, e.g. with herbicides, insecticides, growth regulators, fungicides or else with fertilizers. Mixing the compounds I or the compositions comprising them in the application form as fungicides with other fungicides results in many cases in an expansion of the fungicidal spectrum of activity being obtained.

The following list of fungicides, in conjunction with which the compounds according to the invention can be used, is intended to illustrate the possible combinations but does not limit them:
acylalanines, such as benalaxyl, metalaxyl, ofurace or oxadixyl,
amine derivatives, such as aldimorph, dodine, dodemorph, fenpropimorph, fenpropidin, guazatine, iminoctadine, spiroxamine or tridemorph,
anilinopyrimidines, such as pyrimethanil, mepanipyrim or cyprodinyl,
antibiotics, such as cycloheximide, griseofulvin, kasugamycin, natamycin, polyoxin or streptomycin,
azoles, such as bitertanol, bromoconazole, cyproconazole, difenoconazole, dinitroconazole, epoxiconazole, fenbuconazole, fluquinconazole, flusilazole, flutriafol hexaconazole, imazalil, metconazole, myclobutanil, penconazole, propiconazole, prochloraz, prothioconazole, tebuconazole, triadimefon, triadimenol, triflumizole or triticonazole,
dicarboximides, such as iprodione, myclozolin, procymidone or vinclozolin,
dithiocarbamates, such as ferbam, nabam, maneb, mancozeb, metam, metiram, propineb, polycarbamate, thiram, ziram or zineb,
heterocyclic compounds, such as anilazine, benomyl, boscalid, carbendazim, carboxin, oxycarboxin, cyazofamid, dazomet, dithianon, famoxadone, fenamidone, fenarimol, fuberidazole, flutolanil, furametpyr, isoprothiolane, mepronil, nuarimol, probenazole, proquinazid, pyrifenox, pyroquilon, quinoxyfen, silthiofam, thiabendazole, thifluzamide, thiophanate-methyl, tiadinil, tricyclazole or triforine,
copper fungicides, such as Bordeaux mixture, copper acetate, copper oxychloride or basic copper sulfate,
nitrophenyl derivatives, such as binapacryl, dinocap, dinobuton or nitrophthalisopropyl,
phenylpyrroles, such as fenpiclonil or fludioxonil,
sulfur,
other fungicides, such as acibenzolar-5-methyl, benthiavalicarb, carpropamid, chlorothalonil, cyflufenamid, cymoxanil, diclomezine, diclocymet, diethofencarb, edifenphos, ethaboxam, fenhexamid, fentin acetate, fenoxanil, ferimzone, fluazinam, fosetyl, fosetyl-aluminum, iprovalicarb, hexachlorobenzene, metrafenone, pencycuron, propamocarb, phthalide, tolclofos-methyl, quintozene or zoxamide,
strobilurins, such as azoxystrobin, dimoxystrobin, fluoxastrobin, kresoxim-methyl, metominostrobin, orysastrobin, picoxystrobin, pyraclostrobin or trifloxystrobin,
sulfenic acid derivatives, such as captafol, captan, dichlofluanid, folpet or tolylfluanid,
cinnamides and analogous compounds, such as dimethomorph, flumetover or flumorph.

### SYNTHESIS EXAMPLES

The procedures described in the synthesis examples below were used to prepare further compounds I by appropriate modification of the starting compounds. The compounds thus obtained are listed in table B below, together with physical data.

### Example 1

### 5-Chloro-7-(4-methylpiperidin-1-ylmethyl)-6-(2,4,6-trifluorophenyl)[1,2,4]triazolo[1,5-a]pyrimidine and 5-(4-methylpiperidin-1-yl)-7-(4-methylpiperidin-1-ylmethyl)-6-(2,4,6-trifluorophenyl)[1,2,4]triazolo[1,5-a]pyrimidine

1.1 5-Chloro-6-(2,4,6-trifluorophenyl)[1,2,4]triazolo[1,5-a]pyrimidin-7-carbonitrile

Over a period of 15 minutes, a solution of 1.1 equivalents of tetraethylammonium cyanide in 50 ml of acetonitrile was added with stirring to a solution of 5,7-dichloro-6-(2,4,6-trifluorophenyl)[1,2,4]triazolo[1,5-a]pyrimidine (62.7 mmol) [cf. WO 98/46607] in 300 ml of acetonitrile which had been cooled to 0° C. The reaction mixture was stirred at 0° C. for one hour and then allowed to warm to room temperature over a period of two hours.

The reaction mixture was evaporated to dryness and the solid residue obtained in this manner was then dissolved in ethyl acetate. Insoluble material (tetraethylammonium chloride) was filtered off. The clear solution obtained in this manner was washed in each case three times with 5% strength aqueous citric acid, saturated sodium bicarbonate solution and sodium chloride solution. The organic phase was dried over anhydrous Na₂SO₄, filtered and evaporated to dryness. This gave the title compound in a yield of 94%. The title compound obtained in this manner was used without further purification for the next step.

1.2 5-Chloro-7-methyl-6-(2,4,6-trifluorophenyl)[1,2,4]triazolo[1,5-a]pyrimidine

Tetrahydrofuran was added to a three-necked flask which had been made inert with argon, and the flask was cooled to −60° C. in a dry ice/acetone bath. Using a dropping funnel, 13.4 mmol (3 equivalents) of methylmagnesium chloride in tetrahydrofuran were then added, and the dropping funnel was rinsed with a few mls of tetrahydrofuran. At an internal temperature of the flask of at most −50° C., a solution of 5-chloro-6-(2,4,6-trifluorophenyl)[1,2,4]triazolo[1,5-a]pyrimidin-7-carbonitrile (4.5 mmol) from example 1.1 in tetrahydrofuran was then added dropwise, and the mixture was stirred at this temperature for 1 hour, during which time the reaction was monitored by HPLC/MSD.

The reaction mixture was then poured into a saturated ammonium chloride solution. Diethyl ether was added to the aqueous reaction mixture, the aqueous reaction mixture was extracted and the aqueous phase was washed once with diethyl ether. The combined organic phases were washed twice with 5% strength aqueous citric acid, twice with a saturated sodium bicarbonate solution and then once with a saturated sodium chloride solution. The organic phase was dried over magnesium sulfate, the drying agent was filtered off and the mixture was evaported to dryness, which gave the title compound in a yield of 87%. The title compound was used without further purification for the next reaction step.

1.3 7-Bromomethyl-5-chloro-6-(2,4,6-trifluorophenyl)[1,2,4]triazolo[1,5-a]pyrimidine

1.2 Equivalents of pyridinium tribromide and 1.2 equivalents of pyridine were added to a solution of 5-chloro-7-methyl-6-(2,4,6-trifluorophenyl)[1,2,4]triazolo[1,5-a]pyrimidine (40 mmol) from example 1.2 in 600 ml of tetrahydrofuran. The reaction mixture was stirred at 55° C. for 5 days.

The reaction mixture was concentrated and the solid residue obtained in this manner was taken up in ethyl acetate. The solution was washed twice with 5% strength aqueous citric acid and twice with saturated sodium chloride solution. The mixture was dried over magnesium sulfate, the drying agent was then removed and the mixture was concentrated. The residue obtained gave, after chromatography on silica gel (mobile phase: ethyl acetate/cyclohexane 1:2), the title compound in a yield of 55%.

1.4 5-Chloro-7-(4-methylpiperidin-1-ylmethyl)-6-(2,4,6-trifluorophenyl)[1,2,4]triazolo[1,5-a]pyrimidine and 5-(4-methylpiperidin-1-yl)-7-(4-methylpiperidin-1-yl-methyl)-6-(2,4,6-trifluorophenyl)[1,2,4]triazolo[1,5-a]pyrimidine

1.2 Equivalents of triethylamine and 1 equivalent of 4-methylpiperidine were added to a solution of 7-bromomethyl-5-chloro-6-(2,4,6-trifluorophenyl)[1,2,4]triazolo[1,5-a]pyrimidine (0.4 mmol) from example 1.3 in dichloromethane. The reaction mixture obtained in this manner was stirred at room temperature for 15 hours.

Dilute hydrochloric acid was then added to the reaction mixture, the phases were separated and the organic phase was extracted with a dilute sodium chloride solution. The organic phase was concentrated and the residue was purified by pre-parative HPLC on silica gel RP-18 (Chromolith Speed ROD from Merck KGaA, Germany, 40° C.; mobile phase: acetonitrile with 0.1% by volume of trifluoroacetic acid and 0.1% by volume of a trifluoroacetic acid/water mixture, where the ratio of trifluoroacetic acid/water was changed from 5:95 to 95:5 over a period of 5 minutes). This gave 5-chloro-7-(4-methylpiperidin-1-ylmethyl)-6-(2,4,6-trifluorophenyl)[1,2,4]triazolo[1,5-a]pyrimidine in a yield of 60% and 5-(4-methylpiperidin-1-yl)-7-(4-methylpiperidin-1-ylmethyl)-6-(2,4,6-trifluorophenyl)[1,2,4]triazolo[1,5-a]pyrimidine in a yield of 53%.

### Example 2

### Preparation of 5-methyl-7-(4-methylpiperidin-1-ylmethyl)-6-(2,4,6-trifluorophenyl)[1,2,4]triazolo[1,5-a]pyrimidine (preparation route A)

2.1A Dimethyl 2-[7-(4-methylpiperidin-1-ylmethyl)-6-(2,4,6-trifluorophenyl)[1,2,4]triazolo[1,5-a]pyrimidin-5-yl]malonate

24 Equivalents of dimethyl malonate, 6 equivalents of sodium hydride and 4 Å molecular sieves were initially charged in a reaction vessel, and the mixture was diluted with acetonitrile. The vessel was then made inert using argon gas, the mixture was stirred until the evolution of hydrogen had ceased, and 5-chloro-7-(4-methylpiperidin-1-ylmethyl)-6-(2,4,6-trifluorophenyl)[1,2,4]triazolo[1,5-a]pyrimidine (0.23 mmol) from example 1 were added. The reaction mixture was stirred at 90° C. for 4 hours. The reaction mixture was then centrifuged at 0° C. and 4000 rpm for 10 minutes.

After decanting and concentrating the solution, the residue obtained in this manner was taken up in dichloromethane, and 0.1 N hydrochloric acid was added. The phases were separated and the organic phase was washed with water, dried over sodium sulfate and concentrated, which gave the title compound in a yield of 78%. The title compound was used without further purification for the next step.

2.2A 5-Methyl-7-(4-methylpiperidin-1-ylmethyl)-6-(2,4,6-trifluorophenyl)-[1,2,4]triazolo[1,5-a]pyrimidine

A solution of dimethyl 2-[7-(4-methylpiperidin-1-ylmethyl)-6-(2,4,6-trifluorophenyl)[1,2,4]triazolo[1,5-a]pyrimidin-5-yl]malonate (0.15 mmol) from example 2.1A in concentrated hydrochloric acid was heated at 80° C. for 1 hour.

After cooling, the reaction mixture was concentrated and the solid obtained in this manner was taken up in dichloromethane. The mixture was extracted twice with saturated sodium bicarbonate solution and twice with water. The organic phase was dried over sodium sulfate, the drying agent was filtered off and the mixture was concentrated, which gave the title compound in a yield of 83%.

Preparation Route B:

2.1 B Dimethyl 2-[7-methyl-6-(2,4,6-trifluorophenyl)[1,2,4]triazolo[1,5-a]pyrimidin-5-yl]-malonate

24 Equivalents of dimethyl malonate, 6 equivalents of sodium hydride and 4 Å molecular sieves were initially charged in a reaction vessel, and the mixture was then diluted with acetonitrile. The vessel was then made inert using argon gas, the mixture was stirred until the evolution of hydrogen had ceased, and 5-chloro-7-methyl-6-(2,4,6-trifluorophenyl)[1,2,4]triazolo[1,5-a]pyrimidine (2.68 mmol) from example 1.2 was then added. The reaction mixture was stirred at 90° C. for 3 hours.

The reaction mixture was filtered and the precipitate was washed with acetonitrile. The solution obtained in this manner was concentrated, the residue was taken up in dichloromethane and 0.1 N hydrochloric acid was added. After phase separation, the organic phase was washed with water, dried over sodium sulfate and concentrated, which gave the title compound in a yield of 82%. The title compound was used without further purification for the next step.

2.2B 5,7-Dimethyl-6-(2,4,6-trifluorophenyl)[1,2,4]triazolo[1,5-a]pyrimidine

A solution of dimethyl 2-[7-methyl-6-(2,4,6-trifluorophenyl)[1,2,4]triazolo[1,5-a]-pyrimidin-5-yl]malonate (2.03 mmol) from example 2.1B in concentrated hydrochloric acid was heated at 80° C. for 1 hour.

After cooling the reaction mixture was concentrated and the solid obtained in this manner was taken up in dichloromethane. The mixture was extracted twice with saturated sodium bicarbonate solution and twice with water. The organic phase was dried over sodium sulfate, the drying agent was filtered off and the mixture was concentrated, which gave the title compound in a yield of 79%.

2.3B 7-Bromomethyl-5-methyl-6-(2,4,6-trifluorophenyl)[1,2,4]triazolo[1,5-a]pyrimidine and 5-chloro-7-dibromomethyl-6-(2,4,6-trifluorophenyl)[1,2,4]triazolo[1,5-a]-pyrimidine

1.2 Equivalents of pyridine and 1.25 equivalents of pyridinium tribromide were added to a solution of 5,7-dimethyl-6-(2,4,6-trifluorophenyl)[1,2,4]triazolo[1,5-a]-pyrimidine (2 mmol) from example 2.2B in 25 ml of tetrahydrofuran. The reaction mixture was stirred at 55° C. for 4 days.

After cooling, the reaction mixture was poured into ethyl acetate (250 ml) and extracted 3-4 times with in each case 50 ml of saturated sodium chloride solution.

The combined organic extracts were dried over calcium chloride. The drying agent was filtered off and the solvent was then removed. The oil obtained in this manner was absorbed on 8 g of silica gel and then purified on an 80 g silica gel column (mobile phase: ethyl acetate/cyclohexane (1/1)). This gave 7-bromomethyl-5-methyl-6-(2,4,6-trifluorophenyl)[1,2,4]triazolo[1,5-a]pyrimidine (30.3% yield based on the compound from example 2.2B) and 5-chloro-7-dibromomethyl-6-(2,4,6-trifluorophenyl)[1,2,4]triazolo[1,5-a]pyrimidine (12.9% yield based on the compound from example 2.2B).

2.4B 5-Methyl-7-(4-methylpiperidin-1-ylmethyl)-6-(2,4,6-trifluorophenyl)-[1,2,4]triazolo[1,5-a]pyrimidine

1.1 Equivalents of triethylamine and 1.1 equivalents of 4-methylpiperidine were added to a solution of 7-bromomethyl-5-methyl-6-(2,4,6-trifluorophenyl)-[1,2,4]triazolo[1,5-a]pyrimidine (0.06 mmol) from example 2.3B in dichloromethane. The reaction mixture was then stirred at room temperature for 12 hours.

The reaction mixture was washed twice with diluted sodium chloride solution and dried and the solvent was then removed, which gave the title compound in a yield of 74%.

### Example 3

### Preparation of 5-methoxy-7-(4-methylpiperidin-1-ylmethyl)-6-(2,4,6-trifluorophenyl)[1,2,4]triazolo[1,5-a]pyrimidine

8 mg (0.14 mmol) of sodium methoxide were added to a solution of 50 mg (0.12 mmol) of 5-chloro-7-(4-methylpiperidin-1-ylmethyl)-6-(2,4,6-trifluorophenyl)-[1,2,4]triazolo[1,5-a]pyrimidine from example 1 in 2 ml of methanol. The reaction mixture was then shaken at room temperature for 3 hours.

After concentration of the reaction mixture, the residue obtained in this manner was taken up in dichloromethane. The mixture was extracted in each case once with dilute hydrochloric acid and water. The organic phase was dried and the solvent was removed, which gave 47 mg (0.11 mmol) of the title compound (92% of the theoretical yield). According to HPLC-MS, the purity was 90%.

The compounds of the formula I listed in table B below were prepared according to the procedures described above.

All products were characterized by combined HPLC/mass spectrometry (High Performance Liquid Chromatography Mass Spectrometry). HPLC was carried out using an analytic RP-18 column (Chromolith Speed ROD from Merck KGaA, Germany) which was operated at 40° C. Acetonitrile with 0.1% by volume of a trifluoroacetic acid/water mixture and 0.1% by volume of trifluoroacetic acid served as mobile phase. (The ratio of trifluoroacetic acid/water was changed from 5:95 to 95:5 over a period of 5 minutes). Mass spectrometry was carried out using a quadrupole mass spectrometer with electrospray ionization at 80V in the positive mode.

TABLE B

| | |
|---|---|
| | (I) |
| 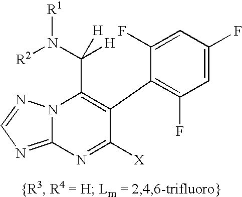 | |

| Example | R¹ | R² | X | min¹⁾/m/z²⁾ |
|---|---|---|---|---|
| 1a | —CH₂CH₂CH(CH₃)CH₂CH₂— | | 4-methyl- | 2.85/458.00 |
| | | | piperidin-1-yl | |
| 1b | —CH₂CH₂CH(CH₃)CH₂CH₂— | | Cl | 2.34/395.00 |
| 2 | —CH₂CH₂CH(CH₃)CH₂CH₂— | | CH₃ | 2.14/375.00 |
| 3 | —CH₂CH₂CH(CH₃)CH₂CH₂— | | OCH₃ | 2.23/391.00 |
| 4 | CH₂CH₃ | CH₂C(CH₃)═CH₂ | Cl | 2.68/395.00 |
| 5 | —CH₂CH₂CH₂CH₂CH₂— | | Cl | 2.16/381.00 |
| 6 | —CH₂CH₂OCH₂CH₂— | | Cl | 2.54/383.00 |
| 7 | —CH₂CH(CH₃)CH(CH₃)CH₂CH₂— | | Cl | 2.53/409.00 |
| 8 | C₆H₅CH₂ | CH₃ | Cl | 2.67/417.00 |
| 9 | —CH(CH₃)CH₂CH₂CH₂— | | Cl | 2.14/381.00 |
| 10 | CH₂CH₃ | CH₂C(CH₃)═CH₂ | CH₃ | 2.19/375.00 |
| 11 | C₆H₅CH₂ | CH₃ | CH₃ | 2.37/397.00 |
| 12 | —CH₂CH₂CH₂CH₂CH₂— | | CH₃ | 1.93/361.00 |
| 13 | C₆H₅CH₂ | CH₃ | OCH₃ | 2.44/413.00 |
| 14 | —CH₂CH₂OCH₂CH₂— | | OCH₃ | 2.08/379.00 |
| 15 | —CH₂CH₂CH₂CH₂— | | CH₃ | 1.83/347.00 |
| 16 | —CH₂CH₂OCH₂CH₂— | | CH₃ | 1.87/363.00 |
| 17 | —CH₂CH(CH₃)CH(CH₃)CH₂CH₂— | | CH₃ | 2.24/389.00 |
| 18 | C₆H₅CH₂ | CH₃ | CH₃ | 2.26/397.00 |
| 19 | CH₂CH₃ | CH₂CH₃ | CH₃ | 1.85/349.00 |
| 20 | —CH₂CH₂CH₂CH₂CH(C(O)OCH₃)— | | CH₃ | 3.03/419.00 |
| 21 | CH(CH₃)CH₂OCH₃ | CH₃ | CH₃ | 1.98/379.00 |
| 22 | CH₂CH(OCH₃)₂ | CH₃ | CH₃ | 2.02/395.00 |
| 23 | —CH(CH₃)CH₂CH₂CH₂— | | CH₃ | 1.94/361.00 |
| 24 | —CH(CH₂CH₃)CH₂CH₂CH₂— | | CH₃ | 2.18/389.00 |
| 25 | —CH₂CH(CH₃)CH₂CH₂CH₂— | | CH₃ | 2.12/375.00 |
| 26 | —CH(CH₃)CH₂CH₂CH₂— | | CH₃ | 2.00/375.00 |
| 27 | 4-methoxybenzyl | CH₃ | CH₃ | 2.37/427.00 |
| 28 | CH₂CH(CH₃)CH(CH₃)₂ | CH₃ | CH₃ | 2.51/391.00 |
| 29 | CH₂CH₂CH₃ | CH₃ | CH₃ | 1.94/349.00 |

¹⁾HPLC retention time in minutes
²⁾m/z of the [M + H]⁺ peak

Examples of the Action Against Harmful Fungi

The fungicidal action of the compounds of formula I was demonstrated by the following tests:

The active compounds were prepared separately or together as a stock solution with 25 mg of active compound which was made up to 10 ml with a mixture of acetone and/or dimethyl sulfoxide (DMSO) and the emulsifier Uniperol® EL (wetting agent having emulsifying and dispersing action based on ethoxylated alkylphenols) in a volume ratio solvent/emulsifier of 99 to 1. The solution was then made up to 100 ml with water. This stock solution was diluted to the active compound concentration stated below using the solvent/emulsifier/water mixture described.

### Use Example 1

### Activity Against Early Blight of Tomato Caused by Alternaria solani

Leaves of potted plants of the cultivar “Goldene Königin” were sprayed to run off point with an aqueous suspension having the concentration of active compounds stated below. The next day, the leaves were infected with an aqueous spore suspension of Alternaria solani in 2% biomalt solution having a density of 0.17×10⁶ spores/ml. The plants were then placed in a water-vapor-saturated chamber at temperatures of between 20 and 22° C. After 5 days, the infection on the untreated, but infected control plants had developed to such an extent that the infection could be determined visually in %.

In this test, the plants which had been treated with 250 ppm of the compound from example 1a, 24, 25 or 26 showed an infection of at most 10%, whereas the untreated plants were 90% infected.

### Use Example 2

### Activity Against Gray Mold on Bell Pepper Leaves Caused by Botrytis cinerea, Protective Application

Bell pepper seedlings of the cultivar “Neusiedler Ideal Elite” were, after 2 to 3 leaves were well developed, sprayed to runoff point with an aqueous suspension having the concentration of active compound stated below. The next day, the treated plants were inoculated with a spore suspension of Botrytis cinerea which contained 1.7×10⁶ spores/ml in a 2% strength aqueous biomalt solution. The test plants were then placed in a dark climatized chamber at 22-24° C. and high atmospheric humidity. After 5 days, the extent of the fungal infection on the leaves could be determined visually in %.

In this test, the plants which had been treated with 250 ppm of the compound from example 24, 25, 26 or 29 showed an infection of at most 15%, whereas the untreated plants were 90% infected.

1. A 7-aminomethyl-1,2,4-triazolo[1,5-a]pyrimidine compound of the formula I 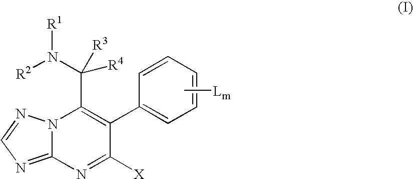
where the index m and the substituents R¹, R², R³, R⁴, X and L are as defined below:
R¹, R² independently of one another are hydrogen, C₁-C₈-alkyl, C₁-C₈-haloalkyl, C₁-C₈-alkoxy, C₃-C₈-cycloalkyl, C₅-C₁₀-bicycloalkyl, C₃-C₈-halocycloalkyl, C₂-C₈-alkenyl, C₄-C₁₀-alkadienyl, C₂-C₈-haloalkenyl, C₃-C₆-cycloalkenyl, C₃-C₆-halocycloalkenyl, C₂-C₈-alkynyl, C₂-C₈-haloalkynyl or phenyl, naphthyl, or a five- or six-membered saturated, partially unsaturated or aromatic heterocycle which contains one, two, three or four heteroatoms from the group consisting of O, N and S,
R¹ and R² together with the nitrogen atom to which they are attached may also form a five- or six-membered heterocyclyl or heteroaryl which is attached via N and may contain one, two or three further heteroatoms from the group consisting of O, N and S as ring members and/or may carry one or more substituents from the group consisting of halogen, C₁-C₆-alkyl, C₁-C₆-haloalkyl, C₂-C₆-alkenyl, C₂-C₆-haloalkenyl, C₁-C₆-alkoxy, C₁-C₆-alkoxycarbonyl, C₁-C₆-haloalkoxy, C₃-C₆-alkenyloxy, C₃-C₆-haloalkenyloxy and/or in which two substituents attached to adjacent ring atoms may be C₁-C₆-alkylene, oxy-C₂-C₄-alkylene or oxy-C₁-C₃-alkyleneoxy;
R¹ and/or R² may carry one, two, three or four identical or different groups R^{a}:
R^{a} is halogen, cyano, nitro, hydroxyl, carboxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₁-C₆-alkylaminocarbonyl, di-C₁-C₆-alkylaminocarbonyl, C₂-C₈-alkenyl, C₄-C₁₀-alkadienyl, C₂-C₈-haloalkenyl, C₃-C₈-cycloalkenyl, C₂-C₆-alkenyloxy, C₃-C₆-haloalkenyloxy, C₂-C₆-alkynyl, C₂-C₆-haloalkynyl, C₃-C₆-alkynyloxy, C₃-C₆-haloalkynyloxy, C₃-C₆-cycloalkoxy, C₃-C₆-cycloalkenyloxy, oxy-C₁-C₃-alkyleneoxy, phenyl, naphthyl, a five-, six-, seven-, eight-, nine- or ten-membered saturated, partially unsaturated or aromatic heterocycle which contains one, two, three or four heteroatoms from the group consisting of O, N and S,
where the aliphatic, alicylic or aromatic groups in R^{a} for their part may be partially or fully halogenated or may carry one, two or three groups R^{b}:
R^{b} is halogen, cyano, nitro, hydroxyl, mercapto, amino, carboxyl, aminocarbonyl, aminothiocarbonyl, alkyl, haloalkyl, alkenyl, alkadienyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothiocarbonyl, where the alkyl groups in these radicals contain 1 to 6 carbon atoms and the alkenyl, alkadienyl or alkynyl groups mentioned in these radicals contain 2 to 8 carbon atoms;
and/or one, two or three of the following radicals:
cycloalkyl, bicycloalkyl, cycloalkoxy, heterocyclyl, heterocyclyloxy, where the cyclic systems contain 3 to 10 ring members; aryl, aryloxy, arylthio, aryl-C₁-C₆-alkoxy, aryl-C₁-C₆-alkyl, hetaryl, hetaryloxy, hetarylthio, where the aryl radicals preferably contain 6, 7, 8, 9 or 10 ring members and the hetaryl radicals 5 or 6 ring members, where the cyclic systems may be partially or fully halogenated or substituted by alkyl or haloalkyl groups;
R³, R⁴ independently of one another are hydrogen, C₁-C₈-alkyl, C₁-C₈-haloalkyl, C₁-C₈-haloalkoxy, C₁-C₈-alkoxy, C₁-C₈-haloalkoxy or C₁-C₈-alkoxy-C₁-C₈-alkyl, where the 6 lastmentioned radicals may carry one, two, three or four identical or different radicals R^{c}, where R^{c} has the meanings given for R^{a};
X is halogen, cyano, a 5- or 6-membered saturated, partially unsaturated or aromatic heterocycle which contains 1, 2 or 3 heteroatoms from the group consisting of O, N and S as ring members, C₁-C₄-alkyl, C₁-C₄-alkoxy, C₂-C₈-alkenyl or C₂-C₈-alkynyl, where the four lastmentioned radicals may be partially or fully halogenated and/or may carry one, two or three substituents selected from the group consisting of nitro, cyano, C₁-C₂-alkoxy, C₁-C₄-alkoxycarbonyl, amino, C₁-C₄-alkylamino and di-C₁-C₄-alkylamino and where the 5- or 6-membered heterocycle may carry one or more substituents from the group consisting of halogen, C₁-C₆-alkyl, C₁-C₆-haloalkyl, C₂-C₆-alkenyl, C₂-C₆-haloalkenyl, C₁-C₆-alkoxy, C₁-C₆-alkoxycarbonyl, C₁-C₆-haloalkoxy, C₃-C₆-alkenyloxy, C₃-C₆-haloalkenyloxy, (exo)-C₁-C₆-alkylene and oxy-C₁-C₃-alkyleneoxy;
L is halogen, cyano, hydroxyl, cyanato (OCN), nitro, C₁-C₈-alkyl, C₂-C₁₀-alkenyl, C₂-C₁₀-alkynyl, C₁-C₆-haloalkyl, C₂-C₁₀-haloalkenyl, C₁-C₆-alkoxy, C₂-C₁₀-alkenyloxy, C₂-C₁₀-alkynyloxy, C₁-C₆-haloalkoxy, C₃-C₆-cycloalkyl, C₃-C₆-cycloalkenyl, C₃-C₆-cycloalkoxy, C₁-C₈-alkoxycarbonyl, C₂-C₁₀-alkenyloxycarbonyl, C₂-C₁₀-alkynyloxycarbonyl, aminocarbonyl, C₁-C₈-alkylaminocarbonyl, di-(C₁-C₈-)-alkylaminocarbonyl, C₁-C₈-alkoximinoalkyl, C₂-C₁₀-alkenyloximinocarbonyl, C₂-C₁₀-alkynyloximinoalkyl, C₁-C₉-alkylcarbonyl, C₂-C₁₀-alkenylcarbonyl, C₂-C₁₀-alkynylcarbonyl, C₃-C₆-cycloalkylcarbonyl, a five-, six-, seven-, eight-, nine- or ten-membered saturated, partially unsaturated or aromatic heterocycle which contains one, two, three or four heteroatoms selected from the group consisting of O, N and S, amino, NR⁵R⁶, NR⁵—(C═O)—R⁶, S(═O)ₙA¹, C(═O)A², C(═S)A², a group —C(S)NR⁷R⁸, a group —C(═N—OR⁹)(NR¹⁰R¹¹) or a group —C(═N—NR¹²R¹³)(NR¹⁴R¹⁵),
where
R⁵, R⁶ independently of one another are selected from the group consisting of hydrogen, C₁-C₆-alkyl, C₂-C₁₀-alkenyl, C₂-C₁₀-alkynyl, C₃-C₆-cycloalkyl and C₃-C₆-cycloalkenyl, where the 5 lastmentioned radicals may be partially or fully halogenated or may carry one, two, three or four radicals selected from the group consisting of cyano, C₁-C₄-alkoximino, C₂-C₄-alkenyloximino, C₂-C₄-alkynyloximino and C₁-C₄-alkoxy;
A¹ is hydrogen, hydroxyl, C₁-C₈-alkyl, C₁-C₈-alkylamino or di-(C₁-C₈-alkyl)amino;
n is 0, 1 or 2;
A² is C₂-C₈-alkenyl, C₁-C₈-alkoxy, C₁-C₆-haloalkoxy, amino or one of the groups mentioned under A¹;
R⁷, R⁸, R⁹, R¹⁰, R¹¹, R¹², R¹³, R¹⁴ and R¹⁵ independently of one another are selected from the group consisting of hydrogen, C₁-C₆-alkyl, C₃-C₆-cycloalkyl, C₂-C₆-alkenyl and C₂-C₆-alkynyl, where the four lastmentioned radicals may have one, two, three, four, five or six radicals R^{a}; or
R⁷ and R⁸, R¹⁰ and R¹¹, R¹² and R¹³ and/or R¹⁴ and R¹⁵ together with the nitrogen atom to which they are attached form a four-, five- or six-membered saturated or partially unsaturated ring which may carry one, two, three or four substituents independently of one another selected from R^{a};
m is 0, 1, 2, 3, 4 or 5;
and the agriculturally acceptable salts of the compounds I.

2. The compound of the formula I according to claim 1 in which at least one of the radicals R¹ or R² is different from hydrogen.

3. The compound of the formula I according to claim 1 or 2 in which R³ and R⁴ independently of one another are hydrogen, C₁-C₄-alkyl or C₁-C₄-alkoxy.

4. The compound of the formula I according to claim 3 in which at least one of the radicals R³ or R⁴ is hydrogen.

5. The compound of the formula I according to claim 1 or 2 in which X is halogen, cyano, C₁-C₄-alkyl, C₁-C₄-haloalkyl, C₁-C₄-alkoxy or C₁-C₂-haloalkoxy.

6. The compound of the formula I according to claim 5 in which X is halogen.

7. The compound of the formula I according to claim 1 or 2 in which m is 1, 2, 3 or 4 and L is selected from the group consisting of halogen, cyano, nitro, C₁-C₆-alkyl, C₁-C₆-haloalkyl, C₁-C₆-alkoxy, a group C(S)A² or a group C(O)A².

8. The compound of the formula I according to claim 7 in which the phenyl group substituted by Lₘ is the group C 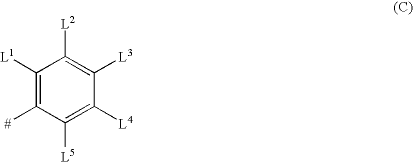
in which # is the point of attachment to the triazolopyrimidine skeleton and
L¹ is fluorine, chlorine, trifluoromethyl or methyl;
L² is hydrogen, chlorine or fluorine;
L³ is hydrogen, CN, nitro, fluorine, chlorine, C₁-C₄-alkyl, C₁-C₄-alkoxy, a group C(S)A^{2a} or a group C(O)A^{2a} in which A^{2a} is C₁-C₄-alkoxy, NH₂, C₁-C₄-alkylamino or di-C₁-C₄-alkylamino;
L⁴ is hydrogen, chlorine or fluorine; and
L⁵ is hydrogen, fluorine, chlorine or C₁-C₄-alkyl.

9. The use of a compound of the formula I according to claim 1 or 2 and an agriculturally acceptable salt thereof for controlling phytopathogenic fungi.

10. A composition for controlling phytopathogenic fungi, which composition comprises at least one compound of the formula I according to claim 1 or 2 and/or an agriculturally acceptable salt of I and at least one solid or liquid carrier.

11. A method for controlling phytopathogenic fungi, which method comprises treating the fungi or the materials, plants, the soil or seed to be protected against fungal attack with an effective amount of at least one compound of the formula I according to claim 1 or 2 and/or an agriculturally acceptable salt of I.
